(19) United States
(12) Patent Application Publication
(10) Pub. No.: US 2010/0310495 A1
(43) Pub. Date: Dec. 9, 2010

(54) PEPTIDES HAVING AFFINITY FOR POLY (BENZYL METHACRYLATE-CO-METHACRYLIC ACID) POTASSIUM SALT COPOLYMERS AND METHODS OF USE

(75) Inventors: EBERHARD SCHNEIDER, DENKTE (DE); GREGOR SCHURMANN, HANNOVER (DE); PETER WAGNER, BRAUNSCHWEIG (DE); HONG WANG, KENNETT SQUARE, PA (US); GORDON MARK COHEN, WYNNEWOOD, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, WILMINGTON, DE (US)

(21) Appl. No.: 12/785,694

(22) Filed: May 24, 2010

Related U.S. Application Data
(60) Provisional application No. 61/184,912, filed on Jun. 8, 2009.

(51) Int. Cl. A61K 8/90; C07K 14/00; A61Q 3/00; A61Q 5/00; A61Q 11/00; A61Q 19/00; C08G 63/91

(52) U.S. Cl. 424/78.03; 530/324; 525/54.1

(57) ABSTRACT

Peptides are provided that have binding affinity for poly(benzyl methacrylate-co-methacrylic acid) potassium salt copolymers (“BzMA-binding peptides”). The BzMA-binding peptides may be used to prepare peptide-based reagents suitable for use in a variety of applications. The peptide-base reagents may be used to couple benefit agents to a poly(benzyl methacrylate-co-methacrylic acid) potassium salt copolymer surface or may be used to couple a benefit agent comprising a poly(benzyl methacrylate-co-methacrylic acid) potassium salt copolymer surface to a target surface, such as a body surface.

This application claims the benefit of U.S. Provisional Patent Application No. 61/184,912 filed Jun. 8, 2009.

### FIELD OF THE INVENTION

The invention relates to peptides having affinity for poly(benzyl methacrylate-co-methacrylic acid) potassium salt copolymers as well as peptide-based reagents comprising at least one of the present polymer-binding peptides.

### BACKGROUND OF THE INVENTION

Benzyl methacrylate (CAS RN:2495-37-6) is a monomer used to produce a variety of materials including, but not limited to polymers, coatings, films, adhesives, inks, dispersants, textile treatments agents, and lubricating oils. Benzyl methacrylate is often reacted with other monomers, such as methacrylic acid, methyl methacrylate or 2-(dimethylamino) ethyl methacrylate to form copolymers useful in a variety of applications (EP0476840 B1 to Chandross et al., Ito et al., Die Makro molekulare Chemie (1969) 291-293; and Achilleos et al., BioMacromol. (2006) 7(12): 3396-3405).

The copolymer poly(benzyl methacrylate-co-methacrylic acid), potassium salt (CAS RN: 676225-08-4; also referred to herein as “BzMA” copolymer) maybe used in any number of coating applications. The BzMA copolymer has been used as a dispersing agent in pigment-based ink jet formulations and to prepare self-dispersing pigments (United States Published Patent Application NOs. 2006-0014855 to House et al.; US2006-0223908 to Szajewski et al.; US2007-0191508 to Nakagawa et al.; US2008-0206465 to Han-Adebekun et al.; and US2005-0090599 to Spinelli, H., each hereby incorporated by reference).

Peptide-based reagents capable of improving the durability of a benefit agent for a given target surface have been described in the art. These reagents may be designed to have at least one region capable of being coupled to the benefit agent surface and at least one region having an affinity for the target surface. The benefit agent surface may be coated with a polymer to facilitate delivery of the benefit agent to the target surface. Peptides having affinity for various polymers have been reported and are described herein. However, peptides and peptide reagents having affinity for BzMA copolymers have not been reported.

The problem to be solved is to provide peptides having affinity for BzMA copolymers (“BzMA-binding peptides”) as well as peptide-based reagents suitable for either (1) coupling a benefit agent comprising a BzMA copolymer coating to a second target surface to deliver a benefit to the second target surface or (2) coupling a first surface comprising BzMA copolymer to a benefit agent.

### SUMMARY OF THE INVENTION

The problem has been solved by the identification of peptides having affinity for poly(benzyl methacrylate-co-methacrylic acid) potassium salt copolymer (“BzMA-binding peptides”). One or more of the BzMA-binding peptides may be used to prepare peptide-based reagents for use in the delivery of at least one benefit agent to a material comprising poly(benzyl methacrylate-co-methacrylic acid) potassium salt copolymer. One or more of the present BzMA-binding peptides may also be used to couple a benefit agent to a surface comprising BzMA copolymer. The peptide reagents may be used to couple a benefit agent comprising BzMA copolymer (the first target surface) to a second target surface. The first and second target surfaces may be the same or different.

In one embodiment, a peptide having affinity for a poly(benzyl methacrylate-co-methacrylic acid) potassium salt copolymer is provided, wherein the peptide comprises an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and 51.

One or more of the present BzMA-binding peptides (“BzMA_BPs”) may be used to prepare peptide-based reagents. As such, peptide-based reagents are also provided having a general structure selected from the group consisting of:

([BzMA_BP]ₙ-[L]ₓ-BA-[L]_{y})ₘ; and

([BzMA_BP]ₙ-[L]ₓ-TBD-[L]_{y})ₘ

wherein:
i) BzMA_BP is a poly(benzyl methacrylate-co-methacrylic acid) potassium salt copolymer-binding peptide;
ii) L is a linker molecule;
iii) BA is at least one benefit agent;
iv) TBD is a target binding domain;
v) x and y independently range from 0 to 10;
vi) n=1 to 10; and
vii) m=1 to 10;

wherein the poly(benzyl methacrylate-co-methacrylic acid) potassium salt copolymer-binding peptide comprises an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and 51. In another embodiment, the BzMA copolymer-binding peptides have an amino acid sequence selected from the group consisting of SEQ ID NOs: 1-45.

In another embodiment, a method for binding a peptide-based reagent to a BzMA copolymer surface is provided comprising:

a) providing at least one peptide-based reagent comprising at least one of the present BzMA-binding peptides; and

b) contacting the peptide-based reagent of (a) with a surface comprising poly(benzyl methacrylate-co-methacrylic acid) potassium salt copolymer whereby the peptide reagent binds to the poly(benzyl methacrylate-co-methacrylic acid) potassium salt copolymer.

The present BzMA-binding peptides and/or peptide-based reagents may be used in personal care compositions to delivery or enhance the durability of a benefit agent to a body surface. As such, a personal care composition comprising one or more of the present BzMA-binding peptides and/or peptide-based reagents is also provided.

In another embodiment, a composition comprising a benefit agent with a surface coating of poly(benzyl methacrylate-co-methacrylic acid) potassium salt copolymer and a peptide having an affinity for said copolymer is also provided.

### BRIEF DESCRIPTION OF THE BIOLOGICAL SEQUENCES

The following sequences comply with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPC and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs: 1-51 are the amino acid sequences of peptides having affinity for a surface comprising BzMA copolymer.

SEQ ID NO: 52 is the amino acid sequence of the N-terminal constant region used in the present display library.

SEQ ID NO: 53 is the amino acid sequence of the C-terminal constant region used in the present display library.

SEQ ID NO: 54 is the nucleic acid sequence of the oligonucleotide portion of the MHA-oligonucleotide linker used in preparing the fusion molecules.

SEQ ID NOs: 55 and 56 are primers.

SEQ ID NO: 57 is the sequencing primer used in Example 2.

SEQ ID NO: 58 is the amino acid sequence of the Caspase-3 cleavage sequence.

SEQ ID NOs: 59-117 are the amino acid sequence of polymer-binding peptides.

SEQ ID NOs: 118-121 are the amino acid sequence of cellulose acetate-binding peptides.

SEQ ID NOs: 122-176 are the amino acid sequences of pigment-binding peptides.

SEQ ID NOs: 177-191 are the amino acid sequence of clay-binding peptides.

SEQ ID NOs: 192-217 are the amino acid sequences of calcium carbonate-binding peptides.

SEQ ID NOs: 218-240 are the amino acid sequences of silica-binding peptides.

SEQ ID NOs: 241-269 are the amino acid sequences of antimicrobial peptides.

SEQ ID NOs: 270-271 are the amino acid sequences of several peptide linkers.

SEQ ID NOs: 272-273 are the amino acid sequences of several peptide bridges.

SEQ ID NOs: 274-490 are examples of peptides having affinity for a body surface; wherein SEQ ID NOs: 274-400 bind to hair; SEQ ID NOs 396-448 bind to skin; SEQ ID NOs: 449-450 bind to nail; SEQ ID NOs: 451-470 bind to tooth pellicle; and SEQ ID NOs: 471-490 bind to tooth enamel.

### DETAILED DESCRIPTION OF THE INVENTION

Provided herein are peptides having strong affinity for BzMA (BzMA-binding peptides) as well as peptide-based reagents comprising at least one of the present BzMA-binding peptides. The peptide-based reagents are useful for coupling a benefit agent to a surface comprising BzMA copolymer or for coupling at least one first surface comprising poly(benzyl methacrylate-co-methacrylic acid) potassium salt copolymer to at least one second target surface. For example, a particulate benefit agent comprising a BzMA copolymer surface, such as a BzMA-coated pigment, can be coupled to a second surface, such as a body surface. The first and second target surface may be the same or different so long as at least one of the surfaces comprises an effective amount of BzMA copolymer.

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an” and “the” should be read to include one or at least one, and the singular word form of the element or component also includes the plural unless the number is obviously meant to be singular.

The term “comprising” means the presence of the stated features, integers, steps, or components as referred to in the claims, but does not preclude the presence or addition of one or more other features, integers, steps, components or groups thereof. The term “comprising” is intended to include embodiments encompassed by the terms “consisting essentially of and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, the terms “polypeptide” and “peptide” will be used interchangeably to refer to a polymer of two or more amino acids joined together by a peptide bond. In one aspect, this term also includes post expression modifications of the polypeptide, for example, glycosylations, acetylations, phosphorylations and the like. Included within the definition are, for example, peptides containing one or more analogues of an amino acid or labeled amino acids and peptidomimetics. In one embodiment, the peptides are comprised of L-amino acids.

As used herein, the term “benzyl methacrylate-co-methacrylic acid potassium salt copolymer” refers to a random or block copolymer of benzyl methacrylate and methacrylic acid that is subsequently treated with potassium hydroxide. The term “BzMA copolymer” or “92/8 copolymer” will be used to refer to benzyl methacrylate-co-methacrylic acid potassium salt copolymer and all other synonyms under the CAS # 676225-08-4. In one embodiment, the copolymer contains 80-98 wt % benzyl methacrylate and 20-2 wt % methacrylic acid, which is partly or completely neutralized with metal or ammonium hydroxide. A copolymer with 85 wt %-95% benzyl methacrylate and 15-5 wt % methacrylic acid is preferred, 87-93 wt % benzyl methacrylate and 3-7 wt % methacrylic acid more preferred, and 91-93 wt % benzyl methacrylate and 3-7 wt % methacrylic acid most preferred (Spinelli, H., supra). A random copolymer prepared by Group Transfer Polymerization (GTP) is most preferred. A molecular weight range 2000-7000 Daltons is preferred, 3500-5500 Daltons more preferred, and 4000-5000 Daltons is most preferred. The carboxylic acid groups are about 50-100% neutralized; 70-100% is preferred, and 90-100% is most preferred. The hydroxides may be chosen from the group of alkali metal hydroxides, ammonium hydroxide, and alkyammonium hydroxides, dialkylammonium hydroxides, and trialkylammonium hydroxides. Potassium and sodium hydroxide are preferred. Potassium hydroxide is most preferred. Small amounts (such as less than 5 weight percent (wt %), preferably no more than 1 wt %, and most preferably less than 0.1 wt %) of optional acrylate or methacrylate comonomers (“optional comonomers”) may be present, including alkyl methacrylates such as methyl methacrylate, ethyl methacrylate, propyl methacrylate, and butyl methacrylate, phenyl methacrylate, phenyl acrylate, benzyl acrylate, and alkyl acrylates such as methyl acrylate, ethyl acrylate, propyl acrylate, and butyl acrylate. This polymer has a sufficient amount of benzyl methacrylate or other hydrophobic comonomer to make it substantially insoluble in water in its unneutralized acid or salt forms. But it has sufficient methacrylic acid and a sufficient degree of neutralization in its salt form to provide dispersibility in water to materials on which it is coated. The low solubility in water ensures that a small portion of the copolymer resides in water and away from the surface of the material. Thus, little of the peptide that has an affinity for this BzMA copolymer is drawn to the aqueous phase and away from the surface of the material on which the copolymer is coated.

As used herein, “BzMA_BP”, “BzMA-BP” or “BzMA-binding peptide” means a peptide having affinity for a BzMA copolymer. In one embodiment, the BzMA_BP is a peptide having strong affinity for the specified range of poly (benzyl methacrylate-co-methacrylic acid) potassium salt copolymers. In one embodiment, the BzMA_BP is a peptide having affinity for BzMA copolymer comprising about 92 wt % benzyl methacrylate and about 8 wt % methacrylic acid (Spinelli, H., supra).

As used herein, the term “peptide finger” will be used to refer to an individual target surface-binding peptide, typically identified by biopanning against a target surface. Peptides having affinity for BzMA by biopanning may be referred to as “BzMA-binding peptides” or BzMA “peptide fingers”.

As used herein, the term “peptide hand” will be used to refer to a binding domain or region comprising 2 or more “peptide fingers” coupled together using one or more optional, independently-selected linkers, wherein the inclusion of at least one peptide linker is preferred.

As used herein, the terms “BzMA hand” and “BzMA-binding domain” will refer to a single chain peptide comprising of at least two BzMA-binding peptides linked together by an optional molecular linker (L) (“linker”) or spacer, wherein the inclusion of a molecular linker is preferred. In one embodiment, the molecular linker is a peptide linker. In another embodiment, the peptide linker ranges in length from 1 to 50 amino acids, preferably 3 to 25 amino acids in length, and may be comprised of various amino acids. In another embodiment, the molecular linker may be comprised of one or more of the amino acids selected from the group consisting of proline, lysine, glycine, alanine, glutamic acid, serine, and combinations thereof.

As used herein, the term “peptide-based reagent” or “peptide reagent” refers to a single chain peptide comprising at least one of the present BzMA-binding peptides having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and 51. In another embodiment, the peptide-based reagent comprises two or more of the present BzMA-binding peptides separated by a molecular linker. The peptide-based reagent may also have at least one region that can be coupled to the benefit agent and/or a region that provides a binding affinity for a second target surface. As such, the peptide-based reagent may used as an interfacial material to couple a benefit agent or an additional target surface (via a target surface-binding domain or “TBD”) to a surface comprised of BzMA copolymer. The benefit agent-binding region may be comprised of at least one benefit agent-binding peptide (i.e., a peptide having affinity for the benefit agent). The benefit agent may be coupled covalently or non-covalently to the present peptide-based reagents. In one embodiment, the benefit agent is coupled non-covalently to the peptide-based reagent. In another embodiment, the benefit agent is coupled to the peptide-based reagent covalently.

As used herein, the terms “coupling” and “coupled” refer to any chemical association and may include both covalent and non-covalent interactions. In one embodiment, the coupling is non-covalent. In another embodiment, the coupling is covalent.

As used herein, the term “bridge”, “peptide bridge”, and “bridging element” will refer to a linear peptide used to join a BzMA-binding domain (the “first domain”) to a peptide domain (the “second domain”) capable of binding to the surface of particulate benefit agent (i.e., covalent or non-covalent coupling) or a second target surface via a target surface-binding domain (TBD). The peptide bridge may range in size from 1 to 60 amino acids in length, preferably 6 to 30 amino acids in length. Examples of peptide bridges are provided as SEQ ID NOs: 272-273.

The term “benefit agent’ is abbreviated as “BA” and is a general term applying to a compound or substance that may be coupled to a surface comprising BzMA copolymer using one of the present BzMA-binding peptides or peptide-based reagents in order to provide a desirable characteristic of the benefit agent to the complex. In the most general sense a benefit agent may be any element, molecule or compound that is not BzMA copolymer. In one embodiment, the benefit agent may be one or more of the BzMA-binding peptides. Benefit agents typically include, but are not limited to, colorants such as pigments and dyes as well as pharmaceuticals, markers, conditioners, fragrances, as well as domains having a defined activity (“active domains” or “AD”) such as enzyme catalysts, and antimicrobial agents, such as antimicrobial peptides.

The term “target binding domain” is abbreviated as “TBD” will refer to a portion or region of the peptide reagent having affinity for a target surface. In one embodiment, the TBD has strong affinity for a target surface. In another embodiment, the present peptide-based reagents will comprise at least one region or domain having strong affinity for a surface comprising BzMA copolymer, wherein the domain having affinity for BzMA copolymer comprises of at least one of the present BzMA-binding peptides; and at least one second region or domain having strong affinity for a benefit agent or another target surface including, but not limited to body surfaces such as hair, skin, nails, teeth, gums, and corneal tissue, as well as other surfaces such as pigments, synthetic polymers, peptides, nucleic acids, conditioning agents, print media, clay, calcium carbonate, silica, and other particulate benefit agents, such as microspheres. In one embodiment, the target binding domain (TBD) is a body surface-binding domain selected from the group consisting of a hair-binding domain, a skin-binding domain, a nail-binding domain, a tooth-binding domain (both tooth pellicle-binding peptides and/or tooth enamel-binding peptides), and domains having affinity for other body surfaces, such as the gums or corneal tissue. Examples of various peptides having affinity various benefit agent surfaces are provided in the present sequence descriptions and the accompanying sequence listing.

The term “body surface” will mean any surface of the human body that may serve as a substrate for the binding of a peptide carrying a benefit agent. Typical body surfaces may include, but are not limited to, hair, skin, nails, teeth (enamel and/or pellicle surfaces), gums, and corneal tissue. In one embodiment, the body surface is selected from the group consisting of hair, skin, nail, tooth enamel, and tooth pellicle.

As used herein, “BSBP” means body surface-binding peptide. A body surface-binding peptide is a peptide having strong affinity for a specified body surface. A body surface-binding peptide is a peptide ranging in size from 7 to 60 amino acids in length that binds with strong affinity to at least one body surface. As used herein, the body surface-binding peptide is selected from the group consisting of hair-binding peptides, skin-binding peptides, nail-binding peptides, and oral cavity surface-binding peptides, such as tooth enamel-binding peptides, and tooth pellicle-binding peptides. In one embodiment, the body surface-binding peptide is selected from the group consisting of a hair-binding peptide, a skin-binding peptide, a nail-binding peptide, and a tooth-binding peptide (enamel or pellicle). Examples of body surface-binding peptides are provided as SEQ ID NOs: 274-490.

As used herein, the term “hair” as used herein refers to human hair, eyebrows, and eyelashes. The term “hair surface” will mean the surface of human hair capable of binding to a hair-binding peptide. As used herein, the term “hair-binding peptide” refers to a peptide that binds with high affinity to hair. Examples of hair-binding peptides are described in U.S. Patent Application Publication NOs. 2005-0226839; 2007-0065387; 2007-0110686; 2007-0196305; U.S. patent application Ser. Nos. 11/877,692 and 11/939,583; U.S. Pat. No. 7,220,405; and published PCT Application No. WO2004/048399. Examples of hair-binding peptides are provided as SEQ ID NOs: 274-400.

The term “skin” as used herein refers to human skin, or pig skin, VITRO-SKIN® and EPIDERM™ which are substitutes for human skin. Skin will generally comprise a layer of epithelial cells and may additionally comprise a layer of endothelial cells.

Examples of skin-binding peptides are described in U.S. Patent Application Publication Nos. 2005-0249682; 2006-0199206; 2007-0065387; and 2007-0110686; U.S. patent application Ser. No. 11/877,692; and published PCT Application No. WO2004/048399.

As used herein, the term “skin-binding peptide” refers to peptides that bind with strong affinity to skin. Examples of skin-binding peptides (“fingers”) have also been reported (U.S. Patent Application Publication NOs. 2007-0274931 and 2007-0249805 and published PCT Patent Application WO 2004/000257). The skin-binding peptides may be linked together to form skin-binding domains (“hands”). Examples of skin-binding peptides are provided as SEQ ID NOs: 396-448.

As used herein, the term “nails” as used herein refers to human fingernails and toenails. As used herein, the term “nail-binding peptide” refers to peptide sequences that bind with strong affinity to nail. Examples of nail-binding peptides are provided as SEQ ID NOs: 449-450. The nail-binding peptides may be linked together to form nail-binding domains (“hands”).

As used herein, the term “oral cavity surface-binding peptide” refers to peptides that bind with strong affinity to teeth, gums, cheeks, tongue, or other surfaces in the oral cavity. As used herein, the term “tooth-binding peptide” will refer to a peptide that binds with high affinity to tooth enamel or tooth pellicle. Examples of tooth-binding peptides (“fingers”) are disclosed in co-pending U.S. Patent Application Publication No. 2008-0280810 and are provided as SEQ ID NOs: 451-490. The tooth-binding fingers may be linked together to form tooth-binding domains. In one embodiment, the oral cavity surface-binding peptide is a peptide that binds with high affinity to a tooth surface.

The term “tooth surface” will refer to a surface comprised of tooth enamel (typically exposed after professional cleaning or polishing) or tooth pellicle (an acquired surface comprising salivary proteins). Hydroxyapatite can be coated with salivary glycoproteins to mimic a natural tooth pellicle surface (tooth enamel is predominantly comprised of hydroxyapatite).

As used herein, the terms “pellicle” and “tooth pellicle” will refer to the thin film (typically ranging from about 1 μm to about 200 μm thick) derived from salivary glycoproteins which forms over the surface of the tooth crown. Daily tooth brushing tends to only remove a portion of the pellicle surface while abrasive tooth cleaning and/or polishing (typically by a dental professional) will exposure more of the tooth enamel surface. Examples of tooth pellicle-binding peptides are provided as SEQ ID NOs: 451-470.

As used herein, the terms “enamel” and “tooth enamel” will refer to the highly mineralized tissue which forms the outer layer of the tooth. The enamel layer is composed primarily of crystalline calcium phosphate (i.e., hydroxyapatite) along with water and some organic material. In one embodiment, the tooth surface is selected from the group consisting of tooth enamel and tooth pellicle. Examples of tooth enamel-binding peptides are provided as SEQ ID NOs: 471-490.

As used herein, the term “pigment” means an insoluble colorant. A wide variety of organic and inorganic pigments alone or in combination may be used. In one embodiment, the pigment is a metal oxide. As used herein, the term “pigment lake” or “lake” refers to a pigment manufactured by precipitating a dye with an inert binder, usually a metallic salt.

As used herein, “Pigment-BP” means pigment-binding peptide. A pigment-binding peptide is a peptide that binds with strong affinity to a specified pigment. Pigment-binding peptides have been reported in the art (U.S. Patent Application Publ. No. 2005-0054752, U.S. Pat. No. 7,285,264 and co-pending U.S. patent application Ser. No. 12/632,827). Examples of pigment-binding peptides are provided as SEQ ID NOs: 122-176. Examples of iron oxide-based pigment binding peptides are provided as SEQ ID NOs: 148-176 (see U.S. patent application Ser. No. 12/632,827).

As used herein, a “polymer” is a natural or synthetic compound of usually high molecular weight consisting of repeated linked units. As used herein, “Poly-BP” means polymer-binding peptide. Examples of peptides that bind with strong affinity to a specified polymer (in addition to the present BzMA copolymer-binding peptides) have been described (U.S. Patent Application Publication NO. 2008-0206809 and U.S. patent application Nos. 11/607,732, 11/607,792, 11/607,723, 11/607,734, 11/607,672, 11/607,673, and 11/514,804). Examples of polymer-binding peptides may include peptides that bind to polymethyl methacrylate (SEQ ID NOs: 59-85), polypropylene (SEQ ID NOs: 86-92), polytetrafluoroethylene (SEQ ID NOs: 93-101), polyethylene (102-108), nylon (SEQ ID NOs: 109-114), and polystyrene (SEQ ID NOs: 115-117).

Additional peptides having strong affinity for their respective surfaces also include, but are not limited to, cellulose acetate-binding peptides (SEQ ID NOs: 118-121); silica-binding peptides (U.S. patent application Ser. No. 12/632,829 and SEQ ID NOs: 218-240); clay-binding peptides (U.S. Patent Application Publication No. US-2007-0249805 and SEQ ID NOs: 177-191); and calcium carbonate-binding peptides (U.S. Patent Application Publication No. 2009-0029902 and SEQ ID NOs: 192-217).

As used herein, an “antimicrobial peptide” is a peptide having the ability to kill microbial cell populations (see U.S. Pat. No. 7,427,656). Examples of antimicrobial peptides are provided as SEQ ID NOs: 241-269.

As used herein, the term “MB₅₀” refers to the concentration of the binding peptide that gives a signal that is 50% of the maximum signal obtained in an ELISA-based binding assay (see Example 9 of U.S. Published Patent Application No. 2005-0226839; hereby incorporated by reference). The MB₅₀ provides an indication of the strength of the binding interaction or affinity of the components of the complex. The lower the value of MB₅₀, the stronger the interaction of the peptide with its corresponding substrate.

As used herein, the terms “binding affinity” and “affinity” refer to the strength of the interaction of a binding peptide (e.g., target surface-binding peptides, target surface-binding domains, and peptide-base reagents) with its respective substrate. The binding affinity may be reported in terms of the MB₅₀ value as determined in an ELISA-based binding assay or as a K_{D} (equilibrium dissociation constant) value, which may be deduced using a methodology, such as surface plasmon resonance (SPR).

As used herein, the term “strong affinity” refers to a binding affinity, as measured as an MB₅₀ value or K_{D} value, of 10⁻⁴ M or less, preferably less than 10⁻⁵ M, more preferably less than 10⁻⁶ M, more preferably less than 10⁻⁷ M, even more preferably less than 10⁻⁸ M, and most preferably less than 10⁻⁹ M.

As used herein, “L” means “molecular linker” or “linker”. The linker may be a peptide or non-peptide-based molecular linker. In one embodiment, the linker is a peptide linker. Peptide linkers separating the BzMA-binding domain from a benefit agent, a benefit agent-binding domain or a target surface-binding domain (TBD) may also be referred to as a peptide “bridge” or “bridging element”. In one embodiment, the peptide linker is 1 to 60 amino acids in length, preferably 3 to 25 amino acids in length. Examples of peptide linkers are provided as SEQ ID NOs: 270-271. The peptide linker or peptide bridge may be a cleavable peptide sequence, such as the Caspase-3 cleavage sequence (SEQ ID NO: 58).

In one embodiment, the benefit agent may be an active domain within (i.e., a subsequence of the peptide reagent) or coupled to the peptide reagent. In one embodiment, the active domain is a portion of the peptide reagent that is not responsible for BzMA copolymer binding but provides additional functionality or benefit. In another embodiment the active domain may have antimicrobial functionality. For example, the peptide reagent may be comprised of at least one of the present BzMA-binding peptides and at least one antimicrobial peptide; whereby coupling of said peptide reagent to a surface comprising BzMA copolymer provides a surface characterized by an enhancement in antimicrobial activity.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Miscellaneous (or as defined herein) | Xaa | X |

### Binding Affinity

The present BzMA-binding peptides exhibit a strong affinity for a surface comprising BzMA copolymer based on their ability to bind to a BzMA copolymer after many rounds of selection under stringent selection conditions. The BzMA copolymer biopanned against in the present examples was comprised of a poly(benzyl methacrylate-co-methacrylic acid) potassium salt polymer having about 92 mol % benzyl methacrylate and about 8 mol % methacrylic acid. The affinity of the peptide for BzMA copolymer can be expressed in terms of the dissociation constant K_{D} or an ELISA-based MB₅₀ value. K_{D} (expressed as molar concentration) corresponds to the concentration of peptide at which the binding site on the target is half occupied, i.e. when the concentration of target with peptide bound (bound target material) equals the concentration of target with no peptide bound. The smaller the dissociation constant, the more tightly bound the peptide is; for example, a peptide with a nanomolar (nM) dissociation constant binds more tightly than a peptide with a micromolar (μM) dissociation constant. In one embodiment, the BzMA-binding peptides have a K_{D} value of 10⁻⁴ M or less, preferably 10⁻⁸ M or less, more preferably 10⁻⁶ M or less, even more preferably 10⁻⁷ M or less, yet even more preferably 10⁻⁸ M or less, and most preferably 10⁻⁹ M or less.

Alternatively, one of skill in the art can also use an ELISA-based assay to calculate a relative affinity of the peptide for the target material (reported as an MB₅₀ value; see present Example 3 and co-owned U.S. Patent Application Publication No. 2005-022683, herein incorporated by reference). As used herein, the term “MB₅₀” refers to the concentration of the binding peptide that gives a signal that is 50% of the maximum signal obtained in an ELISA-based binding assay. The MB₅₀ value provides an indication of the strength of the binding interaction or affinity of the components of the complex. The lower the value of MB₅₀, the stronger the interaction of the peptide with its corresponding substrate. In one embodiment, the MB₅₀ value (reported in terms of molar concentration) for the BzMA-binding peptide is 10⁻⁴ M or less, preferably 10⁻⁸ M or less, more preferably 10⁻⁶ M or less, more preferably 10⁻⁷ M or less, and most preferably 10⁻⁸ M or less.

mRNA-Display and Phage Display
mRNA-Display

Some of the present BzMA-binding peptides were biopanned against poly(benzyl methacrylate-co-methacrylic acid) potassium salt copolymer using mRNA display, an in vitro panning method commonly used for identifying peptides having an affinity for a target material (U.S. Pat. No. 6,258,558). Briefly, a random library of DNA molecules was generated wherein they encode a peptide of a desired length. The length of the peptide within the display library may be to be up to 200 amino acids in length and is typically designed to range from about 7 to about 100 amino acids in length. In one embodiment, the library of peptides may be designed to be about 7 to about 60 amino acids in length, preferably about 7 to about 30 amino acids in length, more preferably about 15 to about 30 amino acids in length, and most preferably about 27 amino acids in length (i.e., a “27-mer” library). Typically, the nucleic acid molecule encoding the peptide includes (in addition to the coding region) appropriate 5′ and 3′ regulatory regions necessary for efficient in vitro transcription and translation. The design of the nucleic acid constructs used for preparing the mRNA-display library is well known to one of skill in the (see WO2005/051985). The nucleic acid molecules can be designed to optionally encode flexible linkers, cleavage sequences, fusion promoting sequences, and identification/purification tags (e.g., poly-A regions, His tags, etc.) to facility purification and/or processing in subsequence steps.

The library of random nucleic acid fragments is transcribed in vitro to produce an mRNA library. The mRNA is isolated and subsequently fused to a linker molecule (i.e., a puromycin-oligonucleotide linker or a puromycin derivative-oligonucleotide linker is used) using techniques well-known in the art (U.S. Pat. No. 6,258,558; U.S. Pat. No. 6,228,994; and Kurz et al., (2000) NAR, 28(18):e83 i-v). In a preferred embodiment, the puromycin-oligonucleotide linker comprises psoralen for rapid and facile preparation of the mRNA-protein fusions (Kurtz et al., supra). The mRNA-puromycin fusion molecules are then translated in vitro whereby the nascent polypeptide is fused (via the puromycin-oligonucleotide linker) to the mRNA (PROFUSION™ molecules; Adnexus Therapeutics, Weltham, Mass.). In this way, the phenotype (peptide) is linked to the corresponding genotype (RNA).

The mRNA-peptide fusion molecules are typically reverse transcribed into a DNA/mRNA-protein fusion molecules prior to affinity selection. The library (often comprising up to 10¹³ different sequences) is contacted with target ligand/material (typically an immobilized target and/or a solid surface). The selection process is carried out in an aqueous medium wherein parameters such as time, temperature, pH, buffer, salt concentration, and detergent concentration may be varied according the stringency of the selection strategy employed. Typically, the temperature of the incubation period ranges from 0° C. to about 40° C. and the incubation time ranges from about 1 to about 24 hours.

Several washing steps are typically used to remove the non-binding/low affinity fusion molecules. The stringency of the washing conditions may be adjusted to select those fusion molecules having the highest affinity for the target material. The high affinity fusion molecules are isolated and then PCR-amplified in order to obtain the nucleic acid sequences encoding the binding peptides. The mRNA-display selection cycle is typically repeated for 3 to 10 cycles in order to select/enrich those fusion molecules comprising peptide sequences exhibiting the highest affinity for the target material.

Error-prone PCR may optionally be incorporated into mRNA-display selection process whereby mutants derived from a previously selected high affinity sequence are used. The process is typically repeated for several cycles in order to obtain the peptides having improved affinity for the target material.

Optionally, any BzMA-binding peptide sequence identified using mRNA-display may be verified using the free peptide. Typically, the nucleic acid molecule encoding the BzMA-binding peptide is cloned and recombinantly expressed in an appropriate microbial host cell, such as E. coli. The free peptide is then isolated and assayed against the targeted material to validate the binding affinity of the peptide sequence.

### Phage Display

Some of the present BzMA-binding peptides were identified using phage display biopanning (see U.S. Pat. No. 5,223,409; U.S. Pat. No. 5,403,484; U.S. Pat. No. 5,571,698; and U.S. Pat. No. 5,837,500). The use of phage display to identify peptides having affinity for a target surface, such as a body surface, has been described by Huang et al., U.S. Pat. No. 7,220,405 and U.S. Patent Application Publication No. 2005-0226839; Estell et al. in WO 01/79479; Murray et al., U.S. Patent Application Publication No. 2002-0098524; Janssen et al., U.S. Patent Application Publication No. 2003-0152976; and Janssen et al., WO 04/048399, to name a few. Phage display libraries are available commercially from companies such as New England BioLabs (Beverly, Mass.).

### Poly(Benzyl Methacrylate-Co-Methacrylic Acid) Potassium Salt Copolymers

BzMA copolymer can be prepared using any number of synthesis techniques, including free radical, anionic and Group Transfer Polymerization (see Spinelli, H., supra). The BzMA copolymer may be a random or block copolymer. In one embodiment, the BzMA copolymer is a random copolymer prepared by Group Transfer Polymerization (GTP). For a detailed discussion of GTP, see Webster et al., J. Am Chem. Soc. 105:5706 (1983) and U.S. Pat. Nos. 4,414,372; 4,417,034; 4,508,880; 4,524,196; 4,581,428; 4,558,795. 4,598,161; 4,605,716; 4,622,372; and 4,656,233; each herein incorporated by reference. While it may sometimes be added in solid form, the copolymer is typically provided as a solution.

In one embodiment, the BzMA copolymer is coated onto another surface, such as metal, metal oxide, pigment, glass, cloth, and the like, using methods known in the art, such as spraying, brushing, dip coating and casting. In one embodiment, copolymer poly(benzyl methacrylate-co-methacrylic acid) is applied as a coating and/or formed into a desired shape (such as a bead) and then treated with potassium hydroxide to form the BzMA copolymer

In another embodiment, the BzMA copolymer is imbedded into the surface of another material, such as another polymer. The copolymer can be applied to any surface as a coating from solution or aqueous dispersion

In a preferred embodiment, the BzMA copolymer is used as a dispersant for pigments or other insoluble particles, including metallic and semiconductor nanoparticles.

### Production of Peptides

The present peptides may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the present peptides may be prepared using recombinant DNA and molecular cloning techniques. Genes encoding the present peptides may be produced in heterologous host cells, particularly in the cells of microbial hosts, as described by Huang et al. (U.S. Patent Application Publication No. 2005-0050656).

Preferred heterologous host cells for expression of the present peptides are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, bacterial, fungal or yeast species such as Aspergillus, Trichoderma, Saccharomyces, Pichia, Phaffia, Kluyveromyces, Candida, Hansenula, Yarrowia, Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. In one embodiment, bacterial host strains include Escherichia, Bacillus, and Pseudomonas. In a preferred embodiment, the bacterial host cell is Escherichia coli.

### Benefit Agents

Benefit agents are any material or substance that may be complexed with the peptide-base reagent comprising one or more of the present BzMA-binding peptides in a manner so as to deliver a benefit at the point where the peptide reagent is attached. A benefit agent may be selected for the purpose of adding the physical, chemical and/or biological properties of said agent to the BzMA copolymer surface.

Benefit agents may be inorganic or organic in nature. Some preferred embodiments include benefit agents that are pigments, conditioners, colorants, antimicrobial agents, and fragrances.

### Conditioners

In one embodiment, a peptide reagent may be used that provides a conditioning effect to a body surface. For example, a peptide reagent may be designed to couple a target surface, such as a body surface, with a conditioning agent comprising a surface of BzMA copolymer. The conditioning agent may be provided or incorporated with a bead, particle, or microsphere comprising a BzMA copolymer surface. Conditioner benefits agents as referred to in discussion of the present invention generally mean benefit agents that provide an improvement to the appearance, texture or quality of the substance they are designed to condition. Conditioner benefit agents may be used with the present invention to condition any substance including but not limited to hair, skin, nail, tooth enamel, tooth pellicle, gums, others tissues of the oral cavity, leather, and upholstery. In the preferred embodiment the present invention is used in combination with a benefit agent that provides a conditioning effect to hair, skin, nails, tooth enamel, and tooth pellicle.

Hair conditioning agents are well known in the art, see for example Green et al. (WO 01/07009) and are available commercially from various sources. Suitable examples of hair conditioning agents include, but are not limited to cationic polymers, such as cationized guar gum, diallyl quaternary ammonium salt/acrylamide copolymers, quaternized polyvinylpyrrolidone and derivatives thereof, and various polyquaternium-compounds; cationic surfactants, such as stearalkonium chloride, centrimonium chloride, and Sapamin hydrochloride; fatty alcohols, such as behenyl alcohol; fatty amines, such as stearyl amine; waxes; esters; nonionic polymers, such as polyvinylpyrrolidone, polyvinyl alcohol, and polyethylene glycol; silicones; siloxanes, such as decamethylcyclopentasiloxane; polymer emulsions, such as amodimethicone; and volumizing agents, such as nanoparticles (e.g., silica nanoparticles and polymer nanoparticles). The preferred hair conditioning agents contain amine or hydroxyl functional groups to facilitate coupling to the hair-binding peptides. Examples of conditioning agents are octylamine (CAS No. 111-86-4), stearyl amine (CAS No. 124-30-1), behenyl alcohol (CAS No. 661-19-8, Cognis Corp., Cincinnati, Ohio), vinyl group terminated siloxanes, vinyl group terminated silicone (CAS No. 68083-19-2), vinyl group terminated methyl vinyl siloxanes, vinyl group terminated methyl vinyl silicone (CAS No. 68951-99-5), hydroxyl terminated siloxanes, hydroxyl terminated silicone (CAS No. 80801-30-5), amino-modified silicone derivatives, [(aminoethyl)amino]propyl hydroxyl dimethyl siloxanes, [(aminoethyl)amino]propyl hydroxyl dimethyl silicones, and alpha-tridecyl-omega-hydroxy-poly(oxy-1,2-ethanediyl) (CAS No. 24938-91-8).

If the present peptide-base reagents are to be used in connection with a hair care composition, such as when the target binding domain (TBD) of the peptide reagent has affinity for hair, an effective amount of the peptide reagent (alone or in a complex with a BzMA copolymer-coated benefit agent) for use in a hair care composition is herein defined as a proportion of from about 0.01% to about 10%, preferably about 0.01% to about 5% by weight relative to the total weight of the composition. Components of a cosmetically acceptable medium for hair care compositions are described by Philippe et al. in U.S. Pat. No. 6,280,747, and by Omura et al. in U.S. Pat. No. 6,139,851 and Cannell et al. in U.S. Pat. No. 6,013,250, each of which is incorporated herein by reference. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hare care compositions may contain one or more conventional cosmetic or dermatological additives or adjuvants including but not limited to, antioxidants, preserving agents, fillers, surfactants, UVA and/or UVB sunscreens, fragrances, thickeners, wetting agents and anionic, nonionic or amphoteric polymers, and dyes or pigments.

Skin conditioning agents may include, but are not limited to, astringents, which tighten skin; exfoliants, which remove dead skin cells; emollients, which help maintain a smooth, soft, pliable appearance; humectants, which increase the water content of the top layer of skin; occlusives, which retard evaporation of water from the skin's surface; and miscellaneous compounds that enhance the appearance of dry or damaged skin or reduce flaking and restore suppleness. Particles comprising BzMA copolymer and a skin conditioning agent may be in conjunction with one of the present peptide-based reagents to couple the condition agent to skin (assuming the peptide reagent also comprises a portion having affinity for skin). Skin conditioning agents are well known in the art, see for example Green et al., supra, and are available commercially from various sources. Suitable examples of skin conditioning agents include, but are not limited to alpha-hydroxy acids, beta-hydroxy acids, polyols, hyaluronic acid, D,L-panthenol, polysalicylates, vitamin A palmitate, vitamin E acetate, glycerin, sorbitol, silicones, silicone derivatives, lanolin, natural oils and triglyceride esters. The skin conditioning agents may also include polysalicylates, propylene glycol (CAS No. 57-55-6, Dow Chemical, Midland, Mich.), glycerin (CAS No. 56-81-5, Proctor & Gamble Co., Cincinnati, Ohio), glycolic acid (CAS No. 79-14-1, DuPont Co., Wilmington, Del.), lactic acid (CAS No. 50-21-5, Alfa Aesar, Ward Hill, Mass.), malic acid (CAS No. 617-48-1, Alfa Aesar), citric acid (CAS No. 77-92-9, Alfa Aesar), tartaric acid (CAS NO. 133-37-9, Alfa Aesar), glucaric acid (CAS No. 87-73-0), galactaric acid (CAS No. 526-99-8), 3-hydroxyvaleric acid (CAS No. 10237-77-1), salicylic acid (CAS No. 69-72-7, Alfa Aesar), and 1,3 propanediol (CAS No. 504-63-2, DuPont Co., Wilmington, Del.). Polysalicylates may be prepared by the method described by White et al. in U.S. Pat. No. 4,855,483, incorporated herein by reference. Glucaric acid may be synthesized using the method described by Merbouh et al. (Carbohydr. Res. (2001) 336:75-78). The 3-hydroxyvaleric acid may be prepared as described by Bramucci et al. in U.S. Pat. No. 6,562,603.

In a number of embodiments the present peptide reagents could be used in a skin care composition (for example, when the peptide reagent comprises a skin-binding domain and a BzMA copolymer binding domain, wherein the benefit agent comprises BzMA copolymer, such as a BzMA copolymer-coated bead or surface coating). Skin care compositions are herein defined as compositions comprising an effective amount of a skin conditioner or a mixture of different skin conditioners in a cosmetically acceptable medium. The uses of these compositions include, but are not limited to, skin care, skin cleansing, make-up, and anti-wrinkle products. If the present invention is desired to be used in connection with a skin care composition an effective amount of the complex for skin care compositions herein defined as a proportion of from about 0.001% to about 10%, preferably about 0.01% to about 5% by weight relative to the total weight of the composition. This proportion may vary as a function of the type of skin care composition. Suitable compositions for a cosmetically acceptable medium are described by Philippe et al., supra. For example, the cosmetically acceptable medium may be an anhydrous composition containing a fatty substance in a proportion generally of from about 10 to about 90% by weight relative to the total weight of the composition, where the fatty phase containing at least one liquid, solid or semi-solid fatty substance. The fatty substance includes, but is not limited to, oils, waxes, gums, and so-called pasty fatty substances. Alternatively, the compositions may be in the form of a stable dispersion such as a water-in-oil or oil-in-water emulsion. Additionally, the compositions may contain one or more conventional cosmetic or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments.

### Colorants.

The term colorant generally refers to a coloring agent. Colorants may be chemically organic or inorganic and may include pigments, lakes or dyes. The colorants may be prepared by covalently attaching at least one of the present BzMA-binding peptides to a coloring agent, either directly or via a linker, using any of the coupling methods known in the art (see for example, U.S. Patent Application Publication No. 2005-0226839).

Pigments are a particularly suitable benefit agent. A wide variety of organic and inorganic pigments alone or in combination may be used. Preferred organic pigments are carbon black, such as Carbon Black FW18, and colored pigments such as CROMOPHTAL® Yellow 131AK (Ciba Specialty Chemicals), SUNFAST® Magenta 122 (Sun Chemical) and SUNFAST® Blue 15:3 (Sun Chemical). Examples of inorganic pigments may include, but are not limited to finely divided metals such as copper, iron, aluminum, and alloys thereof; and metal oxides, such as silica, alumina, and titania. Additional examples of suitable pigments are given by Ma et al. in U.S. Pat. No. 5,085,698, incorporated herein by reference.

Suitable coloring agents that may be used with the present BzMA-binding peptides and/or peptide-base reagents may include, but are not limited to, 4-hydroxypropylamino-3-nitrophenol, 4-amino-3-nitrophenol, 2-amino-6-chloro-4-nitrophenol, 2-nitro-paraphenylenediamine, N,N-hydroxyethyl-2-nitro-phenylenediamine, 4-nitro-indole, Henna, HC Blue 1, HC Blue 2, HC Yellow 4, HC Red 3, HC Red 5, Disperse Violet 4, Disperse Black 9, HC Blue 7, HC Blue 12, HC Yellow 2, HC Yellow 6, HC Yellow 8, HC Yellow 12, HC Brown 2, D&C Yellow 1, D&C Yellow 3, D&C Blue 1, Disperse Blue 3, Disperse violet 1, eosin derivatives such as D&C Red No. 21 and halogenated fluorescein derivatives such as D&C Red No. 27, D&C Red Orange No. 5 in combination with D&C Red No. 21 and D&C Orange No. 10; and pigments, such as D&C Red No. 36 and D&C Orange No. 17, the calcium lakes of D&C Red Nos. 7, 11, 31 and 34, the barium lake of D&C Red No. 12, the strontium lake of D&C Red No. 13, the aluminum lakes of FD&C Yellow No. 5, of FD&C Yellow No. 6, of D&C Red No. 27, of D&C Red No. 21, and of FD&C Blue No. 1, iron oxides, manganese violet, chromium oxide, titanium dioxide, zinc oxide, barium oxide, ultramarine blue, bismuth citrate, and carbon black particles.

### Fragrances.

The BzMA-binding peptides and/or peptide-base reagents may be used to delivery or couple a fragrance to a surface comprising BzMA copolymer. In another embodiment, a particle, bead, or microsphere comprising BzMA copolymer may also be used to delivery a fragrance to a target surface, such as a body surface, provided that the peptide reagent comprises an appropriate target binding domain (TBD), such as a body surface-binding domain.

A fragrance is a complex, compound or element that releases, a substance which may be perceived by the sense of olfaction or chemical detection in any organism, but preferably, in humans. The object sensed or detected may be a part of or the whole of the fragrance benefit agent. In the preferred embodiment the odor is perceived as desirable to humans. However, some uses may combine with a fragrance benefit agent that is repellent to a class of organisms, including a class that contains or is humans. Any known fragrance or odor may be use as a benefit agent. It may be desirable to attach a fragrance benefit agent to the BzMA copolymer-peptide complex by a bond structure or linking molecule that allows the benefit agent to be released, in part or in whole, so that it may be perceived by a sensing organ or chemical detector.

Numerous fragrances, both natural and synthetic, are well known in the art. For example, Secondini (Handbook of Perfumes and Flavors, Chemical Publishing Co., Inc., New York, 1990) describes many of the natural and synthetic fragrances used in cosmetics. Suitable natural fragrances may include, but are not limited to jasmine, narcissus, rose, violet, lavender, mint, spice, vanilla, anise, amber, orange, pine, lemon, wintergreen, rosemary, basil, and spruce. Suitable synthetic fragrances may include, but are no limited to, acetaldehyde, C7 to C16 alcohols, benzyl acetate, butyric acid, citric acid, isobutyl phenyl acetate, linalyl butyrate, malic acid, menthol, phenyl ethyl cinnamate, phenyl propyl formate, tannic acid, terpineol, vanillin, amyl salicylate, benzaldehyde, diphenyl ketone, indole, and the like.

### Single Chain Peptide-Based Reagents

The present peptide reagents comprising at least one of the present BzMA-binding peptides may be used in a composition to couple a benefit agent to surface, film, sheet, particle, bead, or microsphere comprising a surface having BzMA copolymer. In a further embodiment, peptide reagent comprising a target binding domain (TBD) having affinity for a target surface, such as a body surface, may be used to couple a benefit agent comprising BzMA copolymer to the target surface (i.e., the benefit agent comprises a surface of BzMA copolymer capable of binding to the peptide reagent).

In one embodiment, the peptide reagents may contain one or more molecular linkers (L) separating the individual BzMA-binding peptides and/or separating the BzMA-binding peptide(s) or peptide-based reagent from the benefit agent or target binding domain (TBD).

As such, a peptide-based reagent is provided comprising the general structure:

([BzMA_BP]ₙ-[L]ₓ-BA-[L]_{y})ₘ or

([BzMA_BP]ₙ-[L]ₓ-TBD-[L]_{y})ₘ

wherein:
i) BzMA_BP is a poly(benzyl methacrylate-co-methacrylic acid) potassium salt copolymer-binding peptide;
ii) L is a linker molecule;
iii) BA is at least one benefit agent;
iv) TBD is a target binding domain;
v) x and y independently range from 0 to 10;
vi) n=1 to 10; and
vii) m=1 to 10;

wherein the poly(benzyl methacrylate-co-methacrylic acid) potassium salt copolymer binding peptide comprises an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and 51.

It may also be desirable to have multiple binding peptides coupled to the benefit agent to enhance the interaction between the peptide reagent and the surface comprising BzMA copolymer. Either multiple copies of the same binding peptide or a combination of different binding peptides may be used. In the case of large particles, a large number of binding peptides, such as up to about 1,000 peptides, may be coupled to the particle. A smaller number of binding peptides can be coupled to smaller molecules, i.e., up to about 50.

### Linker Molecules

Linker molecules may optionally be used with one or more of the embodiments described herein. The linker may be any of a variety of molecules, such as alkyl chains, phenyl compounds, ethylene glycol, amides, esters and the like. Preferred linkers are hydrophilic and have a chain length from 1 to about 100 atoms, more preferably, from 2 to about 30 atoms. Examples of preferred linkers include, but are not limited to, ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl, and ethyl, propyl, hexyl, steryl, cetyl, and palmitoyl alkyl chains. The linker may be covalently attached to the peptide and the benefit agent using any of the coupling chemistries described above. In order to facilitate incorporation of the linker, a bifunctional cross-linking agent that contains a linker and reactive groups at both ends for coupling to the peptide and the benefit agent may be used. Suitable bifunctional cross-linking agents are well known in the art and may include diamines, such as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyantes, such as hexamethylenediisocyanate; bis oxiranes, such as 1,4 butanediyl diglycidyl ether; dicarboxylic acids, such as succinyldisalicylate; and the like. Heterobifunctional cross-linking agents, which contain a different reactive group at each end, may also be used. Examples of peptide linkers are provided as SEQ ID NOs: 58, 270, and 271.

### Applications of BzMA-Binding Peptides

It will be appreciated by the skilled person that BzMA-binding peptides or peptide reagents comprising at least one of the present BzMA-binding peptides may be used in a multiplicity of formats including as delivery means for delivering benefits agents, in assays for diagnostic applications as well as in materials applications for coating BzMA copolymer surfaces. In one embodiment, a personal care composition comprising one or more of the present BzMA-binding peptides and/or peptide-based reagents is also provided to delivery (or enhance the durability of) a benefit agent to a body surface. In a preferred embodiment, the benefit agent is a BzMA-coated benefit agent. Examples of personal care compositions may include coloring or conditioning compositions for the body surface described herein, such as hair, skin, nail, and/or tooth surfaces.

### EXAMPLES

It should be understood that these examples, while indicating various embodiments of the invention, are provided for illustration purposes. From the above discussion and the examples provided, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “sec” means second(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “pmol” means picomole(s), “mmol” means millimole(s), “μmole” means micromole(s), “g” means gram(s), “μg” means microgram(s), “mg” means milligram(s), “g” means the gravitation constant, “rpm” means revolutions per minute, “pfu” means plaque forming unit, “BSA” means bovine serum albumin, “ELISA” means enzyme-linked immunosorbent assay, “A” means absorbance, “A₄₅₀” means the absorbance measured at a wavelength of 450 nm, “TBS” means Tris-buffered saline, “TBST-X” means Tris-buffered saline containing TWEEN® 20 (CAS# 9005-64-5) where “X” is the weight percent of TWEEN® 20, “vol %” means volume percent, TRITON®-X100 is a detergent having CAS NO: 9002-93-1.

### General Methods:

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989.

All reagents and materials used for the growth and maintenance of bacterial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.), unless otherwise specified.

### Example 1

### Selection of BzMA Copolymer-Binding Peptides Using mRNA-Display Biopanning

The purpose of this Example is to demonstrate enrichment and isolation of BzMA-binding peptides using an mRNA display biopanning method.

mRNA-Display Peptide Libraries:

Methods to make libraries of DNA molecules suitable as starting materials for mRNA-display are well-known in the art (see WO2005/051985). The following procedure was used to identify 27-mer peptides that have strong affinity for a BzMA copolymer target material.

Briefly, a library of random nucleic acid molecules (dsDNA) each molecule encoding a peptide of desired length was generated. A linear peptide library containing 81 nucleotide positions or 27 randomized amino acid positions was used (“p27 library”). The p27 library was designed to include appropriate 5′ and 3′ regions for efficient in vitro transcription, translation, purification, and coupling to the MHA-oligonucleotide linker (MHA is 3′-[α-amino-p-methoxy-hydrocinnamido]-3′-deoxy-adenosine) in the individual molecules.

The DNA encoding the linear peptide library was designed to include a T7 promoter and a tobacco mosaic virus (TMV) translation initiation sequence operably linked to the coding sequence (CDS) (Liu et al., (2000) Methods in Enzymology, 318:268-293). The CDS was designed to encode: (1) a constant N-terminal flaking region comprising a hexa-histidine tag followed by a flexible linker (underlined) sequence (MHHHHHHSGSSSGSGSG; SEQ ID NO: 52), (2) the randomized 27-mer linear peptide, and (3) a constant C-terminal flanking region (TSGGSSGSSLGVASAI; SEQ ID NO: 53) comprising another flexible linker region (bold) and a C-terminal sequence optimized for efficient coupling to the MHA-oligonucleotide linker (double-underlined).

### In Vitro Transcription

Double stranded DNA (dsDNA) as result of the PCR reactions were transcribed into RNA using the RIBOMAX™ Express in vitro transcription kit (Promega Corp., Madison, Wis.). After incubation for at least 45 min at 37° C., DNase I was added and the incubation continued at 37° C. for additional 30 minutes to degrade all template DNA. The reaction mixture was purified by phenol/chloroform extraction. Then free nucleotides were removed by gel filtration using G25 microspin columns (Pharmacia Corp.; Milwaukee, Wis.). The concentration of purified RNA was determined by photometry at 260 nm.

### Library Preparation:

Approximately 10 pmol of highly purified RNA was produced by in vitro transcription from the p27 DNA library and purified after DNase I digestion (by phenol/chloroform extraction and gel filtration, methods described below). The 3′-end of the p27 library RNA was modified by attachment of a MHA-linker molecule (described above) and translated in vitro by means of a rabbit reticulocyte lysate. Covalent fusion products between peptide and coding RNA were purified on magnetic oligo(dT) beads, reverse transcribed, and again purified on a Ni-NTA purification matrix to remove uncoupled RNA and free peptides. About 8 pmol of peptide-RNA-cDNA-fusions were used as input for the first contact with target material during selection round 1.

### Chemical Coupling of RNA and MHA-Oligonucleotide Linker

Purified RNA was annealed (by heat denaturation for 1 minute at 85° C. and cooling down to 25° C. for 10 minutes) with a 1.5-fold excess of MHA-oligonucleotide linker-PEG₂A18 (5′-psoralen-UAG CGG AUG C A₁₈ (PEG-9)₂ CC-MHA [nucleotides shown in italics represent 2′-O-methyl-derivatives] (SEQ ID NO: 54). The covalent coupling was induced by radiation with UV-light (365 nm) for 15 min at room temperature. Aliquots of this reaction mixture before and after irradiation with UV were analyzed on a 6%-TBE-Urea-polyacrylamidgel to control the coupling efficiency (usually at least 60%).

### In Vitro Translation and ³⁵S-Labelling of Peptide-RNA Fusions

Ligated RNA was translated using a rabbit reticulocyte lysate from Promega in presence of 15 μCi ³⁵S-methionine (1000 Ci/mmole). After a 30 min incubation at 30° C., KCl and MgCl₂ were added to a final concentration of 530 mM and 150 mM respectively in order to promote formation of mRNA-peptide-fusions.

### Oligo(dT) Purification

For the purification of peptide-RNA-fusions from translation mixtures molecules were hybridized to magnetic oligo(dT) beads (Miltenyi Biotec; Bergisch Gladbach, Germany) in annealing buffer (100 mM Tris-HCl pH 8.0, 10 mM EDTA, 1 M NaCl and 0.25% TRITON® X-100) for 5 min at 4° C. Beads were separated from the mixture using magnetic-activated cell sorting (MiniMACS®-filtration columns; Miltenyi Biotec), repetitively washed with 100 mM Tris-HCl pH 8.0, 1 M NaCl, 0.25% TRITON® X-100 and finally eluted with water. A sample of this reaction was analyzed on 4-20% Tris/glycine-SDS-PAGE; radioactive bands were visualized using a PhosphoroImager.

### Reverse Transcription (RT)

The RNAs of Oligo(dT)-purified peptide-RNA-fusions were reverse transcribed using SUPERSCRIPT™ II Reverse Transcriptase (Invitrogen, Carlsbad, Calif.) according to the manufacturer's recommendations. RT reactions contained about 1.5-fold excess of 3′-reverse primer. A sample of this reaction was analyzed on 4-20% Tris/glycine-SDS-PAGE; radioactive bands were visualized using a PhosphorImager.

### His-Tag Purification

Reverse transcribed mRNA-peptide-fusion molecules were mixed with Ni-NTA-agarose (QIAGEN; Valencia, Calif.) in HBS buffer (20 mM HEPES (CAS # 7365-45-9) pH 7.0, 150 mM NaCl, 0.025% TRITON® X-100, 100 μg/mL sheared salmon sperm DNA, 1 mg/mL bovine serum albumin (BSA)) and incubated for 60 min at room temperature under gentle shaking. Ni-NTA was then filtrated and washed with HNT buffer (20 mM HEPES pH 7.0, 150 mM NaCl, 0.025% TRITON® X-100) containing 5 mM imidazole. Finally peptide-RNA-cDNA-fusions were eluted with 150 mM imidazole in HNT buffer (20 mM HEPES pH 7.0, 150 mM NaCl, 0.025% TRITON® X-100). A sample of this reaction was analyzed on 4-20% Tris/glycine-SDS-PAGE; radioactive bands were visualized using a PhosphorImager. BSA (final concentration 1 mg/mL) and sheared salmon sperm DNA (final concentration 100 μg/mL) were added to the eluates before contacting with target materials during selection step.

### Selection by Binding to Target Materials and Washing

A. Incubation of Peptide-RNA-cDNA-Fusion Library with Target Material:

Purified peptide-RNA-cDNA-fusions (PROFUSION™ molecules; Adnexus Therapeutics, Waltham, Mass.) after Ni-NTA purification were incubated for 60 minutes at room temperature in 1 mL (final volume) of 20 mM HEPES, pH 7.4, 150 mM NaCl, 1 mg/mL BSA, 100 μg/mL sheared salmon sperm DNA, 0.025% TRITON® X-100 in presence of DEPC-treated (diethylpyrocarbonate), blocked target material. Input activity of purified peptide-RNA-cDNA-fusions was determined by scintillation measurement.

### B. Washing:

Non-binding variants were washed away by one of the following washing procedures listed below:
Washing procedure B: used for washing the target material during selection round 1:
   5×5 sec. each with HNTriton buffer (20 mM HEPES, pH 7.4, 150 mM NaCl, 0.025% TRITON®-X100)
   2×3 min. with 10% shampoo in HNTriton buffer
   3×5 sec. with HNTbuffer
   1×5 sec 150 mM NaCl (for buffer removal before elution with KOH)
Washing procedure C: used for washing of target material during selection round 2-5:
   2×5 sec. each with HNTween buffer (20 mM HEPES, pH 7.4, 150 mM NaCl, 0.5% Tween-20)
   1×5 min. with 10% shampoo in HNTriton buffer
   1×5 sec with HNTween buffer including tube change
   1×5 min with 10% shampoo in HNTriton buffer
   3×5 sec with HNTween buffer; 1 tube change during the third wash
   1×5 sec 150 mM NaCl (for buffer removal before elution with KOH)
Washing procedure E1: used for washing target material in round 6:
   2×5 sec each with HNTween buffer (20 mM HEPES, pH 7.4, 150 mM NaCl, 0.5% TWEEN®-20)
   1×5 min 10% shampoo in HNTriton buffer
   1×5 sec with HNTween buffer including tube change
   4×30 min. with 10% shampoo in HNTriton buffer
   3×5 sec with HNTween buffer; 1 tube change during the third wash
   1×5 sec 150 mM NaCl (for buffer removal before elution with KOH)
Washing procedure E2: used for washing target material in round 7:
   2×5 sec each with HNTween buffer (20 mM HEPES, pH 7.4, 150 mM NaCl, 0.5% TWEEN®-20)
   1×5 min 10% shampoo in HNTriton buffer
   1×5 sec with HNTween buffer including tube change
   2 days, 20×5 min 10% shampoo in HNTriton buffer
   3×5 sec with HNTween buffer; 1 tube change during the third wash
   1×5 sec 150 mM NaCl (for buffer removal before elution with KOH)
Washing procedure E3: used for washing target material in round 8:
   2×5 sec each with HNTween buffer (20 mM HEPES, pH 7.4, 150 mM NaCl, 0.5% TWEEN®-20)
   1×5 min 10% shampoo in HNTriton buffer
   1×5 sec with HNTween buffer including tube change
   8 days, 4×5 min 10% shampoo in HNTriton buffer
   3×5 sec with HNTween buffer; 1 tube change during the third wash
   1×5 sec 150 mM NaCl (for buffer removal before elution with KOH)
Washing procedure E4: used for washing target material in round 9:
   2×5 sec each with HNTween buffer (20 mM HEPES, pH 7.4, 150 mM NaCl, 0.5% TWEEN®-20)
   4×30 min. with 10% shampoo in HNTriton buffer
   1×5 min 10% shampoo in HNTriton buffer
   1×5 sec with HNTween buffer including tube change
   1× overnight with 10% shampoo in HNTriton buffer
   3×5 sec with HNTween buffer; 1 tube change during the third wash
   1×5 sec 150 mM NaCl (for buffer removal before elution with KOH)
      The shampoo used in the above washing procedures was a commercially available hair shampoo having the following composition:

| | |
|---|---|
| Water | 51% |
| Ammonium lauryl sulfate | 20% |
| Sodium lauryl ether sulfate | 15% |
| Cocamidopropyl betaine | 7% |
| Cocamide MEA | 2.5% |
| Miscellaneous minor components** | ~4.5% |

**(e.g. various pH adjusters, preservatives, vitamins, chelating agents, dispersants, lubricants, fragrances, and dyes)

### Comment on Incubation and Washing Conditions:

Normally during mRNA display selections a low detergent concentration is chosen to have low stringent conditions during up to 6 rounds of selection by keeping the detergent concentration at 0.025% TRITON®-X100. However, a higher stringency for the target material was applied from the beginning during incubation and washing (see washing procedures). The applied high concentrations of TWEEN®-20 and shampoo are close to the so called “critical micelle concentration” (CMC) allowing the formation of small micelles which might contain more than one peptide-RNA-cDNA-fusion. Since CMC driven aggregation of peptide-RNA-cDNA-fusions are critical for successful selections, higher concentrations of the detergents described above were not used.

cDNA Elution:

cDNAs of binding variants were eluted by incubation of target material in 50 μL of 100 mM KOH at 60° C. for 30 minutes. After centrifugation, supernatant was removed from target material and transferred into a fresh tube. KOH eluates were subsequently neutralized by addition of 1 μL of 1 M Tris/HCl, pH 7.0 and 3.8 μL of 1 M HCl (per 50 μL 100 mM KOH).

### Polymerase Chain Reaction (PCR):

After elution in KOH and neutralization, the recovered cDNAs were amplified by quantitative PCR with increasing numbers of amplification cycles (12, 15, 18, 21, 24 and 27 cycles). Products were subsequently analyzed by agarose gel electrophoresis over 2% agarose gels. Optimized conditions (minimal cycle number to get good enrichment of DNA of correct length) were then applied for a preparative PCR reaction and controlled again by agarose gel electrophoresis.

Analytical and preparative PCR reactions were performed in presence of 10 mM Tris-HCl (pH 8.8 at 25° C.), 50 mM KCl, 0.08% Nonidet P40, 2 mM MgCl₂, 2.5 mM dNTPs, 1 μM of each forward and reverse primer (5′-TAATACGACTCATAGGGACAATTACTATTTACAA TTACAATG-3′; SEQ ID NO: 55) and (5′-AATTAAATAGCGGATGCTACACCAAGACTAGAACCGCTG-3′; SEQ ID NO: 56), ⅕ volume of neutralized cDNA eluate and 0.05 U/μL Tag polymerase (Promega Corp.). Temperature program of PCR reaction is given below: Initial denaturation: 90 sec at 94° C.; cycling: 15 sec at 94° C. (denaturation), 20 sec at 60° C. (annealing), 30 sec at 72° C. (extension); post treatment: 3 min at 72° C. (post-treatment); hold at 4° C.

Enrichment of cDNA-RNA-Peptide Fusion Molecules Binding to BzMA Copolymer

Ten rounds of selection were conducted and the relative binding of radioactively labeled cDNA-RNA-peptide fusion molecules to the BzMA copolymer target material was measured. The amount of DEPC treated, blocked BzMA copolymer (100 μg per selection round); DEPC-treated pellet.

Round 1 selection used washing procedure B as described above. Rounds 2-9 used various washing procedures with increased washing stringencies (see Table1). The relative amount of enrichment (reported as percent enrichment of binding molecules relative to their respective input signals [activity of cDNA-RNA-peptide fusions before contacting with the target material]) is provided in Table 1.

TABLE 1
Relative binding of radioactive-labeled peptide-RNA-cDNA-
fusions on BzMA (“92Bz/8”) copolymer during increasing
rounds of mRNA display selection:

| | | % Enrichment of |
|---|---|---|
| | | cDNA-RNA-peptide |
| | | fusion molecules |
| Selection | Washing | having affinity for |
| Round | Procedure | BzMA copolymer |
| R1 | B | — |
| R2 | C | 0.013% |
| R3 | C | 1.81% |
| R4 | C | 8.43% |
| R5 | C | 6.55%^{a} |
| R6 | E1 | 5.95% |
| R7 | E2 | 0.51% |
| R8 | E3 | 0.34% |
| R9 | E4 | 2.38%^{a} |

^{a}= processed for sequencing

### Sequencing of 27-mer BzMA-Binding Peptides

The cDNA molecules from the enriched pool of BzMA-binding fusion molecules were isolated and PCR amplified as described above. The sequences of the DNA molecules encoding the BzMA-binding peptides isolated after rounds 5 and 9 of selection were determined (˜21 samples each). The corresponding amino acid sequences of the BzMA-binding peptides are provided in Tables 2.

TABLE 2
Amino Acid Sequences of Selected BzMA-binding
Peptides from mRNA-display

| Peptide | | | SEQ |
|---|---|---|---|
| Sample | Number | | ID |
| Number | Observed | Amino acid sequence | NO: |
| Round 5, | 1 | RRWRVHMMGRFVGLWRQLKEAMRTILV | 1 |
| sample 1 | | | |
| Round 5, | 1 | AFKAAWSKLITRLFKRRWTKMIGHWRP | 2 |
| sample 2 | | | |
| Round 5, | 1 | LWKLSWRPHTRLGRRLKELWVLRGSWW | 3 |
| sample 3 | | | |
| Round 5, | 1 | MSLWAWLRWRAQRRLHKMLGLRTLLAT | 4 |
| sample 4 | | | |
| Round 5, | 1 | VARLGRKAMLWRWLGRLLVRSFKQLTS | 5 |
| sample 5 | | | |
| Round 5, | 1 | MVLRLTKRWVKMTMRLYGLQQRLRALM | 6 |
| sample 6 | | | |
| Round 5, | 1 | AKLLHRFVHVWKKTASLWLSMLKRRLG | 7 |
| sample 7 | | | |
| Round 5, | 1 | LRLVSRPFRWLKLFGAYLKLTMSMHRV | 8 |
| sample 8 | | | |
| Round 5, | 1 | GVRRRVKWRHLIYHTWRRISVWLFRPV | 9 |
| sample 9 | | | |
| Round 5, | 1 | VSILTWQRRRMVSQVHRLMKSWFLWRI | 10 |
| sample 10 | | | |
| Round 5, | 1 | VRTRFMHMMFSMRSRMMAYGVALVRRF | 11 |
| sample 11 | | | |
| Round 5, | 1 | ASWRRHIWLSWMQAVVSALRSTRRWKT | 12 |
| sample 12 | | | |
| Round 5, | 1 | NFNKIQGARAAXRSGTRRKPVR | 13 |
| sample 13 | | | |
| Round 5, | 1 | RLSTIHRLFRRRLGLLPSMVRWIVYVR | 14 |
| sample 14 | | | |
| Round 5, | 1 | WIRRGIERMFGIMRWPARMISSVFRRV | 15 |
| sample 16 | | | |
| Round 5, | 1 | LLVTLWEASRRRRQRLGTLLKWWYGVA | 16 |
| sample 17 | | | |
| Round 5, | 1 | WLLTWFYHRRTPGRRLAREIWSKWLTR | 17 |
| sample 18 | | | |
| Round 5, | 1 | WGIRARMLRRYVKEWKFRLLAHVVSKI | 18 |
| sample 19 | | | |
| Round 5, | 1 | GPWMFWRLHARHYVVQRFMRRLHELFD | 19 |
| sample 20 | | | |
| Round 5, | 1 | RFIKRLFWLGWQRTWVQRLWRRFGSVT | 20 |
| sample 21 | | | |
| Round 9, | 1 | AKGLAYRLLVSTYLWHKRLARLWRGKV | 21 |
| sample 1 | | | |
| Round 9, | 1 | LVRAVRRVLLGPAIRVMQQRFAFWRYV | 22 |
| sample 2 | | | |
| Round 9, | 1 | AFMSHVVRRWMRFHLGLWRSFAAMREV | 23 |
| sample 3 | | | |
| Round 9, | 2^{b} | LVRMVRLMMRSWVRVTHRRSKFHRQMV | 24 |
| samples | | | |
| 4, 9 | | | |
| Round 9, | 1 | SLWRAWTAFRSLRFWPVRQFVAVLVRR | 25 |
| sample 5 | | | |
| Round 9, | 1 | RVLSWVVGRMGRRLNLVRELLRKIMTR | 26 |
| sample 6 | | | |
| Round 9, | 2 | FSGLVLSLWRRHFERQQRRFDRRFGWL | 27 |
| samples | | | |
| 8, 21 | | | |
| Round 9, | 1 | HHRAVAARMRWWALERLRMQWRFLRML | 28 |
| sample 10 | | | |
| Round 9, | 1 | HTRAAWEWLVERARLNVRRRLQFFQWV | 29 |
| sample 11 | | | |
| Round 9, | 1 | IRRFLQAGMGVHRRMRELLRRTLVVFF | 30 |
| sample 12 | | | |
| Round 9, | 1 | LLRRVLGFRYRMHVAVFHRWDKYLSV | 31 |
| sample 13 | | | |
| Round 9, | 1 | XLAAVRLAQVHRRALVRRVVLEGLVT | 32 |
| sample 14 | | | |
| Round 9, | 1 | LATGLRGMVKRIFEVRRVFRKAIWQIM | 33 |
| sample 15 | | | |
| Round 9, | 1 | LVQLQLAGLRRVVAGVGHVRGKFRLL | 34 |
| sample 16 | | | |
| Round 9, | 1 | FGIRKVEPLEAGEDAPALVVVLY | 35 |
| sample 17 | | | |
| Round 9, | 1 | FMAFRRSVHRELIERRFARARFVWAWL | 36 |
| sample 18 | | | |
| Round 9, | 1 | ISRAWRLIRTFHRTVRAFKNHMWSRLL | 37 |
| sample 19 | | | |
| Round 9, | 1b | LVRMVRLMMRSWVRVTHRWSKFHRQMV | 38 |
| sample 20 | | | |

Sequences for Round 5, sample 15 and Round 9, sample 7 were not
identified.
b = Round 9, samples 4, 9, and 20 have identical or nearly identical amino
acid sequences.

Most of the BzMA-binding sequences in Table 2 have at least one region comprising the short amino acid motif Xaa-Arg-Arg; Xaa-Lys-Lys; Xaa-Lys-Arg or Xaa-Arg-Lys; wherein Xaa is typically glycine, valine, leucine, isoleucine, glutamic acid, threonine, methionine, arginine, lysine, histidine, aspartic acid, phenylalanine or tryptophan (bolded).

### Example 2

### Selection of BzMA Copolymer (92Bz/8)-Binding Peptides Using Phage Display

The purpose of this example was to identify peptides that bind poly(benzyl methacrylate-co-methacrylic acid) potassium salt copolymer (92 mol % benzyl methacrylate; 8 mol % methacrylic acid) using standard phage display biopanning method.

The copolymer material was prepared as a solid thick film that was broken down into small particles. For each round of selection, about 20 mg of copolymer particles was used. The particles were incubated in SUPERBLOCK® blocking buffer (Pierce Chemical Company, Rockford, Ill.; Prod. #37535) for 1 hour at room temperature (˜22° C.), followed by 3 washes with TBST (TBS in 0.5% TWEEN®20). Libraries of phage containing random peptide inserts (10¹¹ pfu) from 7 to 20 amino acids were added to each tube. After 60 minutes of incubation at room temperature (˜22° C.) and shaking at 50 rpm, unbound phage were removed by aspirating the liquid out of each well followed by 6 washes with 1.0 mL TBS containing the detergent TWEEN® 20 (TBST, T-0.5%) and 30% of Neutrogena shampoo (Neutrogena Clean Replenishing, Moisturizing Shampoo, Los Angeles, Calif. 90045).

The particle samples were then transferred to a clean tube, and 200 μL of elution buffer consisting of 1 mg/mL BSA in 0.2 M glycine-HCl, pH 2.2, was added to each well and incubated for 10 min to elute the bound phages. Then, 32 μL of neutralization buffer consisting of 1 M Tris-HCl, pH 9.2, was added to each tube. The phage particles, which were in the elution buffer as well as on the particles, were amplified by incubating with diluted E. coli ER2738 cells, from an overnight culture diluted 1:100 in LB medium, at 37° C. for 4.5 h. After this time, the cell culture was centrifuged for 30 seconds and the upper 80% of the supernatant was transferred to a fresh tube, ⅙ volume of PEG/NaCl (20% polyethylene glycol-800, 2.5 M sodium chloride) was added, and the phage was allowed to precipitate overnight at 4° C. The precipitate was collected by centrifugation at 10,000×g at 4° C. and the resulting pellet was resuspended in 1 mL of TBS. This was the first round of amplified stock. The amplified first round phage stock was then titered according to the standard protocol. For the 2^{nd}, 3^{rd} and 4^{th} round of biopanning, more than 2×10¹¹ pfu of phage stock from the previous round was used. The biopanning process was repeated under the same conditions as described above.

After the 4^{th} round of biopanning, 95 random single phage plaque lysates were prepared following the manufacture's instructions (New England Biolabs) and the single stranded phage genomic DNA was purified using the QIAprep Spin M13 Kit (Qiagen, Valencia, Calif.) and sequenced at the DuPont Sequencing Facility using −96 gIII sequencing primer (5′-CCCTCATAGTTAGCGTAACG-3′), given as SEQ ID NO: 57. The displayed peptide is located immediately after the signal peptide of gene III. Based on the peptide sequences, 30 phage candidates showed significant enrichment were selected for further pellicle binding analysis. The Amino acid sequences of selected phage candidates are listed in Table 3.

TABLE 3
Amino Acid Sequences of BzMA Binding Candidates

| Peptide | | |
|---|---|---|
| Sample | | |
| Number | Amino Acid Sequence | SEQ ID NO: |
| 92/8-1 | APWHLSSQYSRT | 39 |
| 92/8-2 | APSPLLVGPPSP | 40 |
| 92/8-3 | DPTQGPVVRIGGMM | 41 |
| 92/8-4 | HTSHKHNNQLTRTM | 42 |
| 92/8-5 | LTLHPQPLDHP | 43 |
| 92/5-6 | NSCPTDYSGVPCMP | 44 |
| 92/8-7 | VTKHLNQISQSY | 45 |

### Example 3

### Characterization of BzMA-Binding Peptides

Enzyme-linked immunosorbent assay (ELISA) was used to evaluate the binding affinity of the select BzMA-binding peptides from Example 2 (biotinylated peptides with C-terminal lysine added are provided as SEQ ID NOs: 46-51).

The identified peptides were synthesized using standard solid phage synthesis method as described in U.S. Pat. No. 7,585,495; herein incorporated by reference. A peptide free sample served as the negative control. All peptides were modified to contain a C-terminal biotinylated lysine residue for detection purposes.

The BzMA copolymer samples comprised of glass beads coated with BzMA resin (100 mg/sample; 92 mol % benzyl methacrylate, 8 mol % methacrylic acid). The BzMA-coated glass beads were incubated in SUPERBLOCK® blocking buffer (Pierce Chemical Company, Rockford, Ill.; Prod. #37535) for 1 hour at room temperature (˜22° C.), followed by 3 washes with TBST (TBS in 0.05% TWEEN® 20). Peptide binding buffer consisting of 20 μM biotinylated peptide in TBST and 1 mg/mL BSA was added to the BzMA-coated glass beads and incubated for 1 hour at room temperature (˜22° C.), followed by 6 TBST washes. Then, the streptavidin-horseradish peroxidase (HRP) conjugate (Pierce Chemical Co., Rockford, Ill.) was added to each well (1.0 μg per well), and incubated for 1 h at room temperature, followed by 6 times of washes with TBST. All samples were transferred to new tubes and the chromogenic agent ABTS (2,2′-azino-bis(3-ethylbenzthiazoline-6-sulphonic acid)) was added. The color development and the absorbance measurements were performed following the manufacturer's protocol. The plates were read at A_{405 nm}. The resulting absorbance values, reported as the mean of at least three replicates, and the standard error of the mean (SEM) are given in Table 4.

TABLE 4
Binding Affinity of Selected Phage Display
Identified BzMA-binding Peptides

| | | Mean | |
|---|---|---|---|
| Peptide | Peptide Sequence | Absorbance | |
| ID NO. | (SEQ ID NO.) | (A₄₀₅ nm) | SEM |
| No peptide | ---------------------- | 0.50 | 0.13 |
| (control) | ---------- | | |
| 92bz/8-2 | APSPLLVGPPSPK | 0.45 | 0.07 |
| | (SEQ ID NO: 46) | | |
| 92bz/8-3 | DPTQGPVVRIGGMMEK | 0.53 | 0.11 |
| | (SEQ ID NO: 47) | | |
| 92bz/8-4 | HTSHKHNNQLTRTMAK | 1.23 | 0.05 |
| | (SEQ ID NO: 48) | | |
| 92bz/8-5 | ALTLHPQPLDHPK | 1.30 | 0.31 |
| | (SEQ ID NO: 49) | | |
| 92bz/8-6 | NSCPTDYSGVPCMPK | 1.44 | 0.10 |
| | (SEQ ID NO: 50) | | |
| 92bz/8-7 | VTKHLNQISQSYK | 1.28 | 0.23 |
| | (SEQ ID NO: 51) | | |

The results demonstrate that many of the BzMA-binding peptides identified using mRNA-display tested had affinity for the BzMA copolymer resin.

What is claimed is:

1. A peptide having affinity for poly(benzyl methacrylate-co-methacrylic acid) potassium salt copolymer, said peptide having an amino acid sequence selected from the group consisting of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and 51.

2. A peptide-based reagent having a general structure selected from the group consisting of:
([BzMA_BP]ₙ-[L]ₓ-BA-[L]_{y})ₘ; and
([BzMA_BP]ₙ-[L]ₓ-TBD-[L]_{y})ₘ
wherein:
i) BzMA_BP is a peptide having affinity for poly(benzyl methacrylate-co-methacrylic acid) potassium salt copolymer;
ii) L is a linker molecule;
iii) BA is at least one benefit agent;
iv) AD is at least one active domain;
v) TBD is a target binding domain;
vi) x and y are independently 0 or 1;
vii) n=1 to 10; and
viii) m=1 to 10;
wherein the BzMA_BP comprises an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and 51.

3. The peptide-based reagent of claim 2 where the poly(benzyl methacrylate-co-methacrylic acid) potassium salt copolymer comprises a random copolymer containing about 85-95 wt % benzyl methacrylate and about 5-15 wt % methacrylic acid and optional comonomers.

4. The peptide-based reagent of claim 2 where the poly(benzyl methacrylate-co-methacrylic acid) potassium salt copolymer comprises a random copolymer prepared by Group Transfer Polymerization containing about 92 wt % benzyl methacrylate and about 8 wt % methacrylic acid.

5. The peptide-based reagent according to claim 2 wherein the linker molecule is a peptide linker ranging form 1 to 50 amino acids in length.

6. The peptide-based reagent according to claim 2 wherein the benefit agent is selected from the group consisting of pharmaceuticals, markers, colorants, conditioners, fragrances, and antimicrobial agents.

7. The peptide-based reagent according to claim 2 wherein the target binding domain is a body surface binding domain comprising at least one peptide having affinity for a body surface selected from the group consisting of hair, skin, nails and teeth.

8. A method for binding a peptide-based reagent to poly(benzyl methacrylate-co-methacrylic acid) potassium salt copolymer comprising:
a) providing the peptide-base reagent according to claim 2; and
b) contacting the peptide-based reagent of (a) with a surface comprising poly(benzyl methacrylate-co-methacrylic acid) potassium salt copolymer whereby the peptide-based reagent binds to the poly(benzyl methacrylate-co-methacrylic acid) potassium salt copolymer.

9. A personal care composition comprising the peptide of claim 1 or the peptide-based reagent of claim 2.

10. A composition comprising a benefit agent with a surface coating of poly(benzyl methacrylate-co-methacrylic acid) potassium salt copolymer and a peptide having an affinity for said copolymer.

11. The composition of claim 10 wherein where the poly(benzyl methacrylate-co-methacrylic acid) potassium salt copolymer comprises a random copolymer containing about 85-95 wt % benzyl methacrylate and about 5-15 wt % methacrylic acid and optional comonomers.

12. The composition of claim 10 where the poly(benzyl methacrylate-co-methacrylic acid) potassium salt copolymer comprises a random copolymer prepared by Group Transfer Polymerization containing about 92 wt % benzyl methacrylate and about 8 wt % methacrylic acid.

13. The composition of claim 10 wherein the peptide comprises an amino acid sequence selected from the group consisting of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and 51.


SEQUENCE LISTING (490 sequences)

SEQ ID NO: 1 - protein, 27 aa, artificial sequence
  BzMA-binding peptide
RRWRVHMMGRFVGLWRQLKEAMRTILV

SEQ ID NO: 2 - protein, 27 aa, artificial sequence
  BzMA-binding peptide
AFKAAWSKLITRLFKRRWTKMIGHWRP

SEQ ID NO: 3 - protein, 27 aa, artificial sequence
  BzMA-binding peptide
LWKLSWRPHTRLGRRLKELWVLRGSWW

SEQ ID NO: 4 - protein, 27 aa, artificial sequence
  BzMA-binding peptide
MSLWAWLRWRAQRRLHKMLGLRTLLAT

SEQ ID NO: 5 - protein, 27 aa, artificial sequence
  BzMA-binding peptide
VARLGRKAMLWRWLGRLLVRSFKQLTS

SEQ ID NO: 6 - protein, 27 aa, artificial sequence
  BzMA-binding peptide
MVLRLTKRWVKMTMRLYGLQQRLRALM

SEQ ID NO: 7 - protein, 27 aa, artificial sequence
  BzMA-binding peptide
AKLLHRFVHVWKKTASLWLSMLKRRLG

SEQ ID NO: 8 - protein, 27 aa, artificial sequence
  BzMA-binding peptide
LRLVSRPFRWLKLFGAYLKLTMSMHRV

SEQ ID NO: 9 - protein, 27 aa, artificial sequence
  BzMA-binding peptide
GVRRRVKWRHLIYHTWRRISVWLFRPV

SEQ ID NO: 10 - protein, 27 aa, artificial sequence
  BzMA-binding peptide
VSILTWQRRRMVSQVHRLMKSWFLWRI

SEQ ID NO: 11 - protein, 27 aa, artificial sequence
  BzMA-binding peptide
VRTRFMHMMFSMRSRMMAYGVALVRRF

SEQ ID NO: 12 - protein, 27 aa, artificial sequence
  BzMA-binding peptide
ASWRRHIWLSWMQAVVSALRSTRRWKT

SEQ ID NO: 13 - protein, 22 aa, artificial sequence
  BzMA-binding peptide
NFNKIQGARAAXRSGTRRKPVR

SEQ ID NO: 14 - protein, 27 aa, artificial sequence
  BzMA-binding peptide
RLSTIHRLFRRRLGLLPSMVRWIVYVR

SEQ ID NO: 15 - protein, 27 aa, artificial sequence
  BzMA-binding peptide
WIRRGIERMFGIMRWPARMISSVFRRV

SEQ ID NO: 16 - protein, 27 aa, artificial sequence
  BzMA-binding peptide
LLVTLWEASRRRRQRLGTLLKWWYGVA

SEQ ID NO: 17 - protein, 27 aa, artificial sequence
  BzMA-binding peptide
WLLTWFYHRRTPGRRLAREIWSKWLTR

SEQ ID NO: 18 - protein, 27 aa, artificial sequence
  BzMA-binding peptide
WGIRARMLRRYVKEWKFRLLAHVVSKI

SEQ ID NO: 19 - protein, 27 aa, artificial sequence
  BzMA-binding peptide
GPWMFWRLHARHYVVQRFMRRLHELFD

SEQ ID NO: 20 - protein, 27 aa, artificial sequence
  BzMA-binding peptide
RFIKRLFWLGWQRTWVQRLWRRFGSVT

SEQ ID NO: 21 - protein, 27 aa, artificial sequence
  BzMA-binding peptide
AKGLAYRLLVSTYLWHKRLARLWRGKV

SEQ ID NO: 22 - protein, 27 aa, artificial sequence
  BzMA-binding peptide
LVRAVRRVLLGPAIRVMQQRFAFWRYV

SEQ ID NO: 23 - protein, 27 aa, artificial sequence
  BzMA-binding peptide
AFMSHVVRRWMRFHLGLWRSFAAMREV

SEQ ID NO: 24 - protein, 27 aa, artificial sequence
  BzMA-binding peptide
LVRMVRLMMRSWVRVTHRRSKFHRQMV

SEQ ID NO: 25 - protein, 27 aa, artificial sequence
  BzMA-binding peptide
SLWRAWTAFRSLRFWPVRQFVAVLVRR

SEQ ID NO: 26 - protein, 27 aa, artificial sequence
  BzMA-binding peptide
RVLSWVVGRMGRRLNLVRELLRKIMTR

SEQ ID NO: 27 - protein, 27 aa, artificial sequence
  BzMA-binding peptide
FSGLVLSLWRRHFERQQRRFDRRFGWL

SEQ ID NO: 28 - protein, 27 aa, artificial sequence
  BzMA-binding peptide
HHRAVAARMRWWALERLRMQWRFLRML

SEQ ID NO: 29 - protein, 27 aa, artificial sequence
  BzMA-binding peptide
HTRAAWEWLVERARLNVRRRLQFFQWV

SEQ ID NO: 30 - protein, 27 aa, artificial sequence
  BzMA-binding peptide
IRRFLQAGMGVHRRMRELLRRTLVVFF

SEQ ID NO: 31 - protein, 27 aa, artificial sequence
  BzMA-binding peptide
LLRRVLGFRYRMHVAVFHRVVDKYLSV

SEQ ID NO: 32 - protein, 26 aa, artificial sequence
  BzMA-binding peptide
XLAAVRLAQVHRRALVRRVVLEGLVT

SEQ ID NO: 33 - protein, 27 aa, artificial sequence
  BzMA-binding peptide
LATGLRGMVKRIFEVRRVFRKAIWQIM

SEQ ID NO: 34 - protein, 26 aa, artificial sequence
  BzMA-binding peptide
LVQLQLAGLRRVVAGVGHVRGKFRLL

SEQ ID NO: 35 - protein, 23 aa, artificial sequence
  BzMA-binding peptide
FGIRKVEPLEAGEDAPALVVVLY

SEQ ID NO: 36 - protein, 27 aa, artificial sequence
  BzMA-binding peptide
FMAFRRSVHRELIERRFARARFVWAWL

SEQ ID NO: 37 - protein, 27 aa, artificial sequence
  BzMA-binding peptide
ISRAWRLIRTFHRTVRAFKNHMWSRLL

SEQ ID NO: 38 - protein, 27 aa, artificial sequence
  BzMA-binding peptide
LVRMVRLMMRSWVRVTHRWSKFHRQMV

SEQ ID NO: 39 - protein, 12 aa, artificial sequence
  BzMA-binding peptide
APWHLSSQYSRT

SEQ ID NO: 40 - protein, 12 aa, artificial sequence
  BzMA-binding peptide
APSPLLVGPPSP

SEQ ID NO: 41 - protein, 14 aa, artificial sequence
  BzMA-binding peptide
DPTQGPVVRIGGMM

SEQ ID NO: 42 - protein, 14 aa, artificial sequence
  BzMA-binding peptide
HTSHKHNNQLTRTM

SEQ ID NO: 43 - protein, 11 aa, artificial sequence
  BzMA-binding peptide
LTLHPQPLDHP

SEQ ID NO: 44 - protein, 14 aa, artificial sequence
  BzMA-binding peptide
NSCPTDYSGVPCMP

SEQ ID NO: 45 - protein, 12 aa, artificial sequence
  BzMA-binding peptide
VTKHLNQISQSY

SEQ ID NO: 46 - protein, 13 aa, artificial sequence
  BzMA-binding peptide
APSPLLVGPPSPK

SEQ ID NO: 47 - protein, 15 aa, artificial sequence
  BzMA-binding peptide
DPTQGPVVRIGGMMK

SEQ ID NO: 48 - protein, 15 aa, artificial sequence
  BzMA-binding peptide
HTSHKHNNQLTRTMK

SEQ ID NO: 49 - protein, 12 aa, artificial sequence
  BzMA-binding peptide
LTLHPQPLDHPK

SEQ ID NO: 50 - protein, 15 aa, artificial sequence
  BzMA-binding peptide
NSCPTDYSGVPCMPK

SEQ ID NO: 51 - protein, 13 aa, artificial sequence
  BzMA-binding peptide
VTKHLNQISQSYK

SEQ ID NO: 52 - protein, 17 aa, artificial sequence
  N terminal constant region
MHHHHHHSGSSSGSGSG

SEQ ID NO: 53 - protein, 16 aa, artificial sequence
  C terminal constant region
TSGGSSGSSLGVASAI

SEQ ID NO: 54 - RNA, 28 nt, artificial sequence
  Synthetic oligonucleotide linker
uagcggaugcaaaaaaaaaaaaaaaaaa

SEQ ID NO: 55 - DNA, 42 bp, artificial sequence
  primer
taatacgactcatagggacaattactatttacaattacaatg

SEQ ID NO: 56 - DNA, 39 bp, artificial sequence
  primer
aattaaatagcggatgctacaccaagactagaaccgctg

SEQ ID NO: 57 - DNA, 20 bp, artificial sequence
  Primer
ccctcatagttagcgtaacg

SEQ ID NO: 58 - protein, 8 aa, Artificial Sequence
  Caspase 3 cleavage site
LESGDEVD

SEQ ID NO: 59 - protein, 12 aa, Artificial sequence
  Polymethylmethacrylate binding peptides
IPWWNIRAPLNA

SEQ ID NO: 60 - protein, 12 aa, Artificial Sequence
  Polymethylmethacrylate-binding peptide
TAVMNVVNNQLS

SEQ ID NO: 61 - protein, 12 aa, Artificial Sequence
  Polymethylmethacrylate-binding peptide
VPWWAPSKLSMQ

SEQ ID NO: 62 - protein, 12 aa, Artificial Sequence
  Polymethylmethacrylate-binding peptide
MVMAPHTPRARS

SEQ ID NO: 63 - protein, 12 aa, Artificial Sequence
  Polymethylmethacrylate-binding peptide
TYPNWAHLLSHY

SEQ ID NO: 64 - protein, 7 aa, Artificial Sequence
  Polymethylmethacrylate-binding peptide
TPWWRIT

SEQ ID NO: 65 - protein, 7 aa, Artificial Sequence
  Polymethylmethacrylate-binding peptide
DLTLPFH

SEQ ID NO: 66 - protein, 7 aa, Artificial Sequence
  Polymethylmethacrylate-binding peptide
GTSIPAM

SEQ ID NO: 67 - protein, 7 aa, Artificial Sequence
  Polymethylmethacrylate-binding peptide
HHKHVVA

SEQ ID NO: 68 - protein, 7 aa, Artificial Sequence
  Polymethylmethacrylate-binding peptide
HHHKHFM

SEQ ID NO: 69 - protein, 7 aa, Artificial Sequence
  Polymethylmethacrylate-binding peptide
HHHRHQG

SEQ ID NO: 70 - protein, 7 aa, Artificial Sequence
  Polymethylmethacrylate-binding peptide
HHWHAPR

SEQ ID NO: 71 - protein, 12 aa, Artificial Sequence
  PMMA-binding peptide
APWHLSSQYSGT

SEQ ID NO: 72 - protein, 14 aa, Artificial Sequence
  PMMA-binding peptide
GYCLRVDEPTVCSG

SEQ ID NO: 73 - protein, 15 aa, Artificial Sequence
  PMMA-binding peptide
HIHPSDNFPHKNRTH

SEQ ID NO: 74 - protein, 20 aa, Artificial Sequence
  PMMA-binding peptide
HTHHDTHKPWPTDDHRNSSV

SEQ ID NO: 75 - protein, 20 aa, Artificial Sequence
  PMMA-binding peptide
PEDRPSRTNALHHNAHHHNA

SEQ ID NO: 76 - protein, 15 aa, Artificial Sequence
  PMMA-binding peptide
TPHNHATTNHHAGKK

SEQ ID NO: 77 - protein, 15 aa, Artificial Sequence
  PMMA-binding peptide
EMVKDSNQRNTRISS

SEQ ID NO: 78 - protein, 12 aa, Artificial Sequence
  PMMA-binding peptide
HYSRYNPGPHPL

SEQ ID NO: 79 - protein, 12 aa, Artificial Sequence
  PMMA-binding peptide
IDTFYMSTMSHS

SEQ ID NO: 80 - protein, 20 aa, Artificial Sequence
  PMMA-binding peptide
PMKEATHPVPPHKHSETPTA

SEQ ID NO: 81 - protein, 12 aa, Artificial Sequence
  PMMA-binding peptide
YQTSSPAKQSVG

SEQ ID NO: 82 - protein, 15 aa, Artificial Sequence
  PMMA-binding peptide
HLPSYQITQTHAQYR

SEQ ID NO: 83 - protein, 20 aa, Artificial Sequence
  PMMA-binding peptide
TTPKTTYHQSRAPVTAMSEV

SEQ ID NO: 84 - protein, 15 aa, Artificial Sequence
  PMMA-binding peptide
DRIHHKSHHVTTNHF

SEQ ID NO: 85 - protein, 12 aa, Artificial Sequence
  PMMA-binding peptide
WAPEKDYMQLMK

SEQ ID NO: 86 - protein, 12 aa, Artificial sequence
  Polypropylene-binding peptides
TSDIKSRSPHHR

SEQ ID NO: 87 - protein, 12 aa, Artificial Sequence
  Polypropylenebinding peptide
HTQNMRMYEPWF

SEQ ID NO: 88 - protein, 7 aa, Artificial Sequence
  Polypropylene-binding peptide
LPPGSLA

SEQ ID NO: 89 - protein, 12 aa, Artificial Sequence
  Polypropylene-binding peptide
MPAVMSSAQVPR

SEQ ID NO: 90 - protein, 12 aa, Artificial Sequence
  Polypropylene-binding peptide
NQSFLPLDFPFR

SEQ ID NO: 91 - protein, 12 aa, Artificial Sequence
  Polypropylene-binding peptide
SILSTMSPHGAT

SEQ ID NO: 92 - protein, 12 aa, Artificial Sequence
  Polypropylene-binding peptide
SMKYSHSTAPAL

SEQ ID NO: 93 - protein, 12 aa, Artificial sequence
  Polytetrafluoroethylene-binding peptides
ESSYSWSPARLS

SEQ ID NO: 94 - protein, 12 aa, Artificial Sequence
  Polytetrafluoroethylene-binding peptide
GPLKLLHAWWQP

SEQ ID NO: 95 - protein, 7 aa, Artificial Sequence
  Polytetrafluoroethylene-binding peptide
NALTRPV

SEQ ID NO: 96 - protein, 7 aa, Artificial Sequence
  Polytetrafluoroethylene-binding peptide
SAPSSKN

SEQ ID NO: 97 - protein, 12 aa, Artificial Sequence
  Polytetrafluoroethylene-binding peptide
SVSVGMKPSPRP

SEQ ID NO: 98 - protein, 12 aa, Artificial Sequence
  Polytetrafluoroethylene-binding peptide
SYYSLPPIFHIP

SEQ ID NO: 99 - protein, 12 aa, Artificial Sequence
  Polytetrafluoroethylene-binding peptide
TFTPYSITHALL

SEQ ID NO: 100 - protein, 12 aa, Artificial Sequence
  Polytetrafluoroethylene-binding peptide
TMGFTAPRFPHY

SEQ ID NO: 101 - protein, 12 aa, Artificial Sequence
  Polytetrafluoroethylene-binding peptide
TNPFPPPPSSPA

SEQ ID NO: 102 - protein, 12 aa, Artificial sequence
  Polyethylene-binding peptides
HNKSSPLTAALP

SEQ ID NO: 103 - protein, 12 aa, Artificial Sequence
  Polyethylene-binding peptide
LPPWKHKTSGVA

SEQ ID NO: 104 - protein, 12 aa, Artificial Sequence
  Polyethylene-binding peptide
LPWWLRDSYLLP

SEQ ID NO: 105 - protein, 12 aa, Artificial Sequence
  Polyethylene-binding peptide
VPWWKHPPLPVP

SEQ ID NO: 106 - protein, 12 aa, Artificial Sequence
  Polyethylene-binding peptide
HHKQWHNHPHHA

SEQ ID NO: 107 - protein, 12 aa, Artificial Sequence
  Polyethylene-binding peptide
HIFSSWHQMWHR

SEQ ID NO: 108 - protein, 12 aa, Artificial Sequence
  Polyethylene-binding peptide
WPAWKTHPILRM

SEQ ID NO: 109 - protein, 7 aa, Artificial sequence
  Nylon-binding peptides
KTPPTRP

SEQ ID NO: 110 - protein, 7 aa, Artificial Sequence
  Nylon-binding peptide
VINPNLD

SEQ ID NO: 111 - protein, 7 aa, Artificial Sequence
  Nylon-binding peptide
KVWIVST

SEQ ID NO: 112 - protein, 7 aa, Artificial Sequence
  Nylon-binding peptide
AEPVAML

SEQ ID NO: 113 - protein, 7 aa, Artificial Sequence
  Nylon-binding peptide
AELVAML

SEQ ID NO: 114 - protein, 7 aa, Artificial Sequence
  Nylon-binding peptide
HSLRLDW

SEQ ID NO: 115 - protein, 13 aa, Artificial sequence
  Polystyrene-binding peptide
TSTASPTMQSKIR

SEQ ID NO: 116 - protein, 12 aa, Artificial Sequence
  Polystyrene-binding peptide
KRNHWQRMHLSA

SEQ ID NO: 117 - protein, 12 aa, Artificial Sequence
  Polystyrene-binding peptide
SHATPPQGLGPQ

SEQ ID NO: 118 - protein, 20 aa, artificial sequence
  cellulose acetate-binding peptide
ATTPPSGKAAAHSAARQKGN

SEQ ID NO: 119 - protein, 15 aa, artificial sequence
  cellulose acetate-binding peptide
DTIHPNKMKSPSSPL

SEQ ID NO: 120 - protein, 20 aa, artificial sequence
  cellulose aceteate-binding peptide
NGNNHTDIPNRSSYTGGSFA

SEQ ID NO: 121 - protein, 15 aa, ARTIFICIAL SEQUENCE
  cellulose acetate-binding peptide
SDETGPQIPHRRPTW

SEQ ID NO: 122 - protein, 7 aa, Artificial Sequence
  Pigment-binding peptide
MPPPLMQ

SEQ ID NO: 123 - protein, 7 aa, Artificial Sequence
  Pigment-binding peptide
FHENWPS

SEQ ID NO: 124 - protein, 12 aa, Artificial Sequence
  Pigment-binding peptide
RTAPTTPLLLSL

SEQ ID NO: 125 - protein, 12 aa, Artificial Sequence
  Pigment-binding peptide
WHLSWSPVPLPT

SEQ ID NO: 126 - protein, 7 aa, Artificial Sequence
  Pigment-binding peptide
PHARLVG

SEQ ID NO: 127 - protein, 7 aa, Artificial Sequence
  Pigment-binding peptide
NIPYHHP

SEQ ID NO: 128 - protein, 7 aa, Artificial Sequence
  Pigment-binding peptide
TTMPAIP

SEQ ID NO: 129 - protein, 7 aa, Artificial Sequence
  Pigment-binding peptide
HNLPPRS

SEQ ID NO: 130 - protein, 12 aa, Artificial Sequence
  Pigment-binding peptide
AHKTQMGVRQPA

SEQ ID NO: 131 - protein, 12 aa, Artificial Sequence
  Pigment-binding peptide
ADNVQMGVSHTP

SEQ ID NO: 132 - protein, 12 aa, Artificial Sequence
  Pigment-binding peptide
AHNAQMGVSHPP

SEQ ID NO: 133 - protein, 12 aa, Artificial Sequence
  Pigment-binding peptide
ADYVGMGVSHRP

SEQ ID NO: 134 - protein, 12 aa, Artificial Sequence
  Pigment-binding peptide
SVSVGMKPSPRP

SEQ ID NO: 135 - protein, 7 aa, Artificial Sequence
  Pigment-binding peptide
YPNTALV

SEQ ID NO: 136 - protein, 7 aa, Artificial Sequence
  Pigment-binding peptide
VATRIVS

SEQ ID NO: 137 - protein, 12 aa, Artificial Sequence
  Pigment-binding peptide
HSLKNSMLTVMA

SEQ ID NO: 138 - protein, 7 aa, Artificial Sequence
  Pigment-binding peptide
NYPTQAP

SEQ ID NO: 139 - protein, 7 aa, Artificial Sequence
  Pigment-binding peptide
KCCYSVG

SEQ ID NO: 140 - protein, 12 aa, Artificial Sequence
  Pigment-binding peptide
RHDLNTWLPPVK

SEQ ID NO: 141 - protein, 12 aa, artificial sequence
  Pigment-binding peptide
EISLPAKLPSAS

SEQ ID NO: 142 - protein, 12 aa, Artificial Sequence
  Pigment-binding peptide
SDYVGMRPSPRH

SEQ ID NO: 143 - protein, 12 aa, Artificial Sequence
  Pigment-binding peptide
SDYVGMRLSPSQ

SEQ ID NO: 144 - protein, 12 aa, Artificial Sequence
  Pigment-binding peptide
SVSVGIQPSPRP

SEQ ID NO: 145 - protein, 12 aa, Artificial Sequence
  Pigment-binding peptide
YVSVGIKPSPRP

SEQ ID NO: 146 - protein, 12 aa, Artificial Sequence
  Pigment-binding peptide
YVCEGIHPCPRP

SEQ ID NO: 147 - protein, 12 aa, artificial sequence
  synthetic pigment binding peptide
WAPEKDHMQLMK

SEQ ID NO: 148 - protein, 12 aa, artificial sequence
  Iron oxide-binding peptide
WAPEKDYMQLMK

SEQ ID NO: 149 - protein, 20 aa, artificial sequence
  Iron oxide-binding peptide
CPLDTPTHKTKHEYKTRCRH

SEQ ID NO: 150 - protein, 18 aa, artificial sequence
  Iron oxide-binding peptide
DHDHPRLHKRQEKSEHLH

SEQ ID NO: 151 - protein, 20 aa, artificial sequence
  Iron oxide-binding peptide
DSHHNHHKQDSRPQHRKTPN

SEQ ID NO: 152 - protein, 15 aa, artificial sequence
  Iron oxide-binding peptide
EGGNAPHHKPHHRKH

SEQ ID NO: 153 - protein, 18 aa, artificial sequence
  Iron oxide-binding peptide
HDSHRPLTQHGHRHSHVP

SEQ ID NO: 154 - protein, 18 aa, artificial sequence
  Iron oxide-binding peptide
HDSNHCSHSTRRPNCART

SEQ ID NO: 155 - protein, 15 aa, artificial sequence
  Iron oxide-binding peptide
ATRVDNTPASNPPSL

SEQ ID NO: 156 - protein, 15 aa, artificial sequence
  Iron oxide-binding peptide
DGIKPFHLMTPTLAN

SEQ ID NO: 157 - protein, 18 aa, artificial sequence
  Iron oxide-binding peptide
DITPPGSTHHRKPHRHQH

SEQ ID NO: 158 - protein, 15 aa, artificial sequence
  Iron oxide-binding peptide
DNLWPQPLNVEDDRY

SEQ ID NO: 159 - protein, 18 aa, artificial sequence
  Iron oxide-binding peptide
ENEKHRHNTHEALHSHFK

SEQ ID NO: 160 - protein, 20 aa, artificial sequence
  Iron oxide-binding peptide
GAIWPASSALMTEHNPTDNH

SEQ ID NO: 161 - protein, 15 aa, artificial sequence
  Iron oxide-binding peptide
GDTNQDTVMWYYTVN

SEQ ID NO: 162 - protein, 15 aa, artificial sequence
  Iron oxide-binding peptide
HNGPYGMLSTGKIHF

SEQ ID NO: 163 - protein, 15 aa, artificial sequence
  Iron oxide-binding peptide
LDGGYRDTPDNYLKG

SEQ ID NO: 164 - protein, 15 aa, artificial sequence
  Iron oxide-binding peptide
LHTKTENSHTNMKTT

SEQ ID NO: 165 - protein, 20 aa, artificial sequence
  Iron oxide-binding peptide
NAQYDPPTLNKGAVRKAAST

SEQ ID NO: 166 - protein, 15 aa, artificial sequence
  Iron oxide-binding peptide
NGNNHTDIPNRSSYT

SEQ ID NO: 167 - protein, 18 aa, artificial sequence
  Iron oxide-binding peptide
QSTNHHHPHAKHPRVNTH

SEQ ID NO: 168 - protein, 15 aa, artificial sequence
  Iron oxide-binding peptide
SNNDYVGTYPATAIQ

SEQ ID NO: 169 - protein, 15 aa, artificial sequence
  Iron oxide-binding peptide
STQHNLHDRNIYFVS

SEQ ID NO: 170 - protein, 20 aa, artificial sequence
  Iron oxide-binding peptide
TANNKTPAGAPNAAVGLAQR

SEQ ID NO: 171 - protein, 15 aa, artificial sequence
  Iron oxide-binding peptide
TEPTRISNYRSIPND

SEQ ID NO: 172 - protein, 18 aa, artificial sequence
  Iron oxide-binding peptide
THNPREHARHHHHNEYKH

SEQ ID NO: 173 - protein, 15 aa, artificial sequence
  Iron oxide-binding peptide
THPPCWYETNCIVQE

SEQ ID NO: 174 - protein, 20 aa, artificial sequence
  Iron oxide-binding peptide
TTNPHKPASHHHDHRPALRH

SEQ ID NO: 175 - protein, 15 aa, artificial sequence
  Iron oxide-binding peptide
WLVADNATDGHSHQK

SEQ ID NO: 176 - protein, 15 aa, artificial sequence
  Iron oxide-binding peptide
YTDSMSDQTPEFAKY

SEQ ID NO: 177 - protein, 27 aa, artificial sequence
  Clay-binding peptide
GHGSPSNSHHGSKKCDMGNSRAKCKRL

SEQ ID NO: 178 - protein, 27 aa, artificial sequence
  Clay-binding peptide
SDRHNLRNSWSISRHCRRKQGRCLPAH

SEQ ID NO: 179 - protein, 27 aa, artificial sequence
  Clay-binding peptide
KKSNKGHHPSSKGKGPPWSEWDKKNGP

SEQ ID NO: 180 - protein, 27 aa, artificial sequence
  Clay-binding peptide
KKSNKGPHPSSKGKGPPWSEWDKKNGP

SEQ ID NO: 181 - protein, 22 aa, artificial sequence
  Clay-binding peptide
VGRHHSKAKQKRPHGGKGQNKN

SEQ ID NO: 182 - protein, 22 aa, artificial sequence
  Clay-binding peptide
VGRHHPKAKQKRPHGGKGQNKN

SEQ ID NO: 183 - protein, 17 aa, artificial sequence
  Clay-binding peptide
GRRPRARGRSRRGSTKT

SEQ ID NO: 184 - protein, 19 aa, artificial sequence
  Clay-binding peptide
LGVIRNHVVRGRRHHQHVR

SEQ ID NO: 185 - protein, 27 aa, artificial sequence
  Clay-binding peptide
QPGRPTEVHPELVRKSAYLVNPSEDIR

SEQ ID NO: 186 - protein, 27 aa, artificial sequence
  Clay-binding peptide
HRSEKPKNVKYKRGYWERGNQKKHGPG

SEQ ID NO: 187 - protein, 27 aa, artificial sequence
  Clay-binding peptide
GSHKRRGSYALLRTRGVGRQAELEHLL

SEQ ID NO: 188 - protein, 27 aa, artificial sequence
  Clay-binding peptide
VGEKPRRKSKGAKAKKARTKEEKLPKN

SEQ ID NO: 189 - protein, 27 aa, artificial sequence
  Clay-binding peptide
NKGHKQSGSPRHSNKKEKKTQQKRGQP

SEQ ID NO: 190 - protein, 27 aa, artificial sequence
  Clay-binding peptide
HWGSQHKTGLRNHKRSRRDSLGKRGTD

SEQ ID NO: 191 - protein, 27 aa, artificial sequence
  Clay-binding peptide
KGWGSSSGPPGLTGKALGKGRLKPKKK

SEQ ID NO: 192 - protein, 27 aa, artificial sequence
  Calcium carbonate binding peptide
RNNKGSKKVDDKRRKTVHNTKSRAKYS

SEQ ID NO: 193 - protein, 27 aa, artificial sequence
  Calcium carbonate binding peptide
RNNKGSKKVDDKRRKTVHNTKSRAKHS

SEQ ID NO: 194 - protein, 27 aa, artificial sequence
  Calcium carbonate binding peptide
RDNKGSKKVDDKRRKTVHNTKSRAKYS

SEQ ID NO: 195 - protein, 27 aa, artificial sequence
  Calcium carbonate binding peptide
RNNKGSKKVDDKRRKTVHSTKSRAKYS

SEQ ID NO: 196 - protein, 27 aa, artificial sequence
  Calcium carbonate binding peptide
RNNKGSRKVDDKRRKTVHNTKSRAKYS

SEQ ID NO: 197 - protein, 27 aa, artificial sequence
  Calcium carbonate binding peptide
RNNKGSKKADDKRRKTVHSTKSRAKYS

SEQ ID NO: 198 - protein, 27 aa, artificial sequence
  Calcium carbonate binding peptide
RNNKGSKKVDDKRRKAVHNKKSRAKYS

SEQ ID NO: 199 - protein, 27 aa, artificial sequence
  Calcium carbonate binding peptide
RNNKGSKKVDDKRRKTVHNTRSRAKYS

SEQ ID NO: 200 - protein, 27 aa, artificial sequence
  Calcium carbonate binding peptide
RNNKGSKKVDDKRRKTVHNTKSRAKFS

SEQ ID NO: 201 - protein, 27 aa, artificial sequence
  Calcium carbonate binding peptide
QRRKLRHPKEKWFGWSEKKVIKKWSRK

SEQ ID NO: 202 - protein, 27 aa, artificial sequence
  Calcium carbonate binding peptide
QRRKFRHPKEKWFGWSEKKVIKXNGRP

SEQ ID NO: 203 - protein, 27 aa, artificial sequence
  Calcium carbonate binding peptide
HKRLVQNKPHRTRKIEGWIKHMVKRQH

SEQ ID NO: 204 - protein, 27 aa, artificial sequence
  Calcium carbonate binding peptide
TRGHIMRPCWIGAMKQGVKKKRTPGWR

SEQ ID NO: 205 - protein, 27 aa, artificial sequence
  Calcium carbonate binding peptide
WKVKRRMVTRTYEFMGKKPCMMLTKRL

SEQ ID NO: 206 - protein, 26 aa, artificial sequence
  Calcium carbonate binding peptide
KKSNKGHHSKAKQKRPHGGKAQNKNT

SEQ ID NO: 207 - protein, 27 aa, artificial sequence
  Calcium carbonate binding peptide
RAHKERFVVRQIGRSQGYKTWQCVRVA

SEQ ID NO: 208 - protein, 27 aa, artificial sequence
  Calcium carbonate binding peptide
SQKPKGHKVKVVVKLCKRPYWRMLNTA

SEQ ID NO: 209 - protein, 27 aa, artificial sequence
  Calcium carbonate binding peptide
NHGCPVNWKVXNPPRGWQRLNHCKWWN

SEQ ID NO: 210 - protein, 27 aa, artificial sequence
  Calcium carbonate binding peptide
RNSRHKEWRRYKRTHVHSHEFYHVECW

SEQ ID NO: 211 - protein, 27 aa, artificial sequence
  Calcium carbonate binding peptide
HRSEKPKNVNYKRGYWERGNQKKHGPG

SEQ ID NO: 212 - protein, 27 aa, artificial sequence
  Calcium carbonate binding peptide
HERTRRGKPDRQKTTHEKRRQGLWIFM

SEQ ID NO: 213 - protein, 27 aa, artificial sequence
  Calcium carbonate binding peptide
PWGTNKRQKHKVHEAKALKKSLWYSNS

SEQ ID NO: 214 - protein, 27 aa, artificial sequence
  Calcium carbonate binding peptide
RRGVVLCHTHRNKRIRLAYSVTKKAWA

SEQ ID NO: 215 - protein, 27 aa, artificial sequence
  Calcium carbonate binding peptide
ERIRWRRLSAEIRAHKWSVLKFRLSCM

SEQ ID NO: 216 - protein, 27 aa, artificial sequence
  Calcium carbonate binding peptide
KTKEKKKEVKLHKKSLSLVLLADLWRL

SEQ ID NO: 217 - protein, 27 aa, artificial sequence
  Calcium carbonate binding peptide
LGKKHKQHSKVGHGKLSTRFLRRSKLF

SEQ ID NO: 218 - protein, 20 aa, artificial sequence
  Silica-binding peptide
AEAKRHPVVPLHEQHGHHEL

SEQ ID NO: 219 - protein, 18 aa, artificial sequence
  Silica-binding peptide
APQTWNRPHPGHPNVHTR

SEQ ID NO: 220 - protein, 20 aa, artificial sequence
  Silica-binding peptide
ATTPPSGKAAAHSAARQKGN

SEQ ID NO: 221 - protein, 20 aa, artificial sequence
  Silica-binding peptide
DGRPDNPKHQQSYNRQLPRQ

SEQ ID NO: 222 - protein, 20 aa, artificial sequence
  Silica-binding peptide
DHNNRQHAVEVRENKTHTAR

SEQ ID NO: 223 - protein, 20 aa, artificial sequence
  Silica-binding peptide
GPEPRALNPKRHMDPATQIR

SEQ ID NO: 224 - protein, 15 aa, artificial sequence
  Silica-binding peptide
HDHHQTHNVLHGMKK

SEQ ID NO: 225 - protein, 18 aa, artificial sequence
  Silica-binding peptide
HHDRAEPRGMAATLAQTI

SEQ ID NO: 226 - protein, 20 aa, artificial sequence
  Silica-binding peptide
HHNHMTGADNPIFHNNTAHR

SEQ ID NO: 227 - protein, 18 aa, artificial sequence
  Silica-binding peptide
HNHAQMLRPEPTGISHKN

SEQ ID NO: 228 - protein, 20 aa, artificial sequence
  Silica-binding peptide
HTNDNGQSTPRRDPPAFQRK

SEQ ID NO: 229 - protein, 18 aa, artificial sequence
  Silica-binding peptide
HTNHHYDQKMHGPLPTPY

SEQ ID NO: 230 - protein, 20 aa, artificial sequence
  Silica-binding peptide
LNSMSDKHHGHQNTATRNQH

SEQ ID NO: 231 - protein, 20 aa, artificial sequence
  Silica-binding peptide
MHKPNNPDTHRSTPSPLGKS

SEQ ID NO: 232 - protein, 18 aa, artificial sequence
  Silica-binding peptide
NFPVYDTTHHGGHRSKLH

SEQ ID NO: 233 - protein, 20 aa, artificial sequence
  Silica-binding peptide
NVHPQSENTNTTRPHKSTQR

SEQ ID NO: 234 - protein, 18 aa, artificial sequence
  Silica-binding peptide
QHGMHSPNLGARMNATPH

SEQ ID NO: 235 - protein, 20 aa, artificial sequence
  Silica-binding peptide
RPNDTHHPGKCDTHAVCHQT

SEQ ID NO: 236 - protein, 20 aa, artificial sequence
  Silica-binding peptide
SHLMHVKAPTDQASTRNRFD

SEQ ID NO: 237 - protein, 20 aa, artificial sequence
  Silica-binding peptide
SSSTPPNSPKHSKYNVWTSP

SEQ ID NO: 238 - protein, 17 aa, artificial sequence
  Silica-binding peptide
VHQTTPQHKDAVNLPRK

SEQ ID NO: 239 - protein, 20 aa, artificial sequence
  Silica-binding peptide
WHSSEGQYKKPNNHRQYHTG

SEQ ID NO: 240 - protein, 15 aa, artificial sequence
  Silica-binding peptide
YKHERHYSQPLKVRH

SEQ ID NO: 241 - protein, 17 aa, Artificial Sequence
  antimicrobial peptide
PKGLKKLLKGLKKLLKL

SEQ ID NO: 242 - protein, 16 aa, Artificial Sequence
  antimicrobial peptide
KGLKKLLKGLKKLLKL

SEQ ID NO: 243 - protein, 16 aa, Artificial Sequence
  antimicrobial peptide
KGLKKLLKLLKKLLKL

SEQ ID NO: 244 - protein, 14 aa, Artificial Sequence
  antimicrobial peptide
LKKLLKLLKKLLKL

SEQ ID NO: 245 - protein, 12 aa, Artificial Sequence
  antimicrobial peptide
LKKLLKLLKKLL

SEQ ID NO: 246 - protein, 17 aa, Artificial Sequence
  antimicrobial peptide
VAKKLAKLAKKLAKLAL

SEQ ID NO: 247 - protein, 13 aa, Artificial Sequence
  antimicrobial peptide
FAKLLAKALKKLL

SEQ ID NO: 248 - protein, 16 aa, Artificial Sequence
  antimicrobial peptide
KGLKKGLKLLKKLLKL

SEQ ID NO: 249 - protein, 16 aa, Artificial Sequence
  antimicrobial peptide
KGLKKLLKLGKKLLKL

SEQ ID NO: 250 - protein, 16 aa, Artificial Sequence
  antimicrobial peptide
KGLKKLGKLLKKLLKL

SEQ ID NO: 251 - protein, 16 aa, Artificial Sequence
  antimicrobial peptide
KGLKKLLKLLKKGLKL

SEQ ID NO: 252 - protein, 16 aa, Artificial Sequence
  antimicrobial peptide
KGLKKLLKLLKKLGKL

SEQ ID NO: 253 - protein, 19 aa, Artificial Sequence
  antimicrobial peptide
FALALKALKKLKKALKKAL

SEQ ID NO: 254 - protein, 17 aa, Artificial Sequence
  antimicrobial peptide
FAKKLAKLAKKLAKLAL

SEQ ID NO: 255 - protein, 13 aa, Artificial Sequence
  antimicrobial peptide
FAKLLAKLAKKLL

SEQ ID NO: 256 - protein, 15 aa, Artificial Sequence
  antimicrobial peptide
FAKKLAKLALKLAKL

SEQ ID NO: 257 - protein, 10 aa, Artificial Sequence
  antimicrobial peptide
FAKKLAKKLL

SEQ ID NO: 258 - protein, 13 aa, Artificial Sequence
  antimicrobial peptide
FAKLLAKLAKKVL

SEQ ID NO: 259 - protein, 13 aa, Artificial Sequence
  antimicrobial peptide
KYKKALKKLAKLL

SEQ ID NO: 260 - protein, 12 aa, Artificial Sequence
  antimicrobial peptide
FALLKALLKKAL

SEQ ID NO: 261 - protein, 14 aa, Artificial Sequence
  antimicrobial peptide
KRLFKKLKFSLRKY

SEQ ID NO: 262 - protein, 14 aa, Artificial Sequence
  antimicrobial peptide
KRLFKKLLFSLRKY

SEQ ID NO: 263 - protein, 14 aa, Artificial Sequence
  antimicrobial peptide
LLLFLLKKRKKRKY

SEQ ID NO: 264 - protein, 36 aa, H. cecropia
KWKLFKKIEKVGQNIRDGIIKAGPAVAWGQATQIAK

SEQ ID NO: 265 - protein, 23 aa, Xenopus
GIGKFLHSAKKFGKAFVGEIMNS

SEQ ID NO: 266 - protein, 22 aa, Xenopus
GIGKFLKKAKKFGKAFVKILKK

SEQ ID NO: 267 - protein, 12 aa, Bos taurus
RLCRIVVIRVCR

SEQ ID NO: 268 - protein, 13 aa, Bos sp.
ILPWKWPWWPWRR

SEQ ID NO: 269 - protein, 24 aa, Homo sapiens
DSHAKRHHGYKRKFHEKHHSHRGY

SEQ ID NO: 270 - protein, 33 aa, artificial sequence
  synthetic linker
EPEPEPEPIPEPPKEAPVVIEKPKPKPKPKPKP

SEQ ID NO: 271 - protein, 21 aa, artificial sequence
  synthetic peptide linker
GKGKGKGKGKGKGKGKGKGKG

SEQ ID NO: 272 - protein, 8 aa, artificial sequence
  synthetic peptide bridge
GSGGGGSP

SEQ ID NO: 273 - protein, 16 aa, artificial sequence
  synthetic peptide bridge
GSGGGGSPGSGGGGSP

SEQ ID NO: 274 - protein, 12 aa, artificial sequence
  synthetic hair-binding peptide
RVPNKTVTVDGA

SEQ ID NO: 275 - protein, 12 aa, artificial sequence
  Synthetic construct
DRHKSKYSSTKS

SEQ ID NO: 276 - protein, 12 aa, artificial sequence
  Synthetic construct
KNFPQQKEFPLS

SEQ ID NO: 277 - protein, 12 aa, artificial sequence
  Synthetic construct
QRNSPPAMSRRD

SEQ ID NO: 278 - protein, 12 aa, artificial sequence
  Synthetic construct
TRKPNMPHGQYL

SEQ ID NO: 279 - protein, 12 aa, artificial sequence
  Synthetic construct
KPPHLAKLPFTT

SEQ ID NO: 280 - protein, 12 aa, artificial sequence
  Synthetic construct
NKRPPTSHRIHA

SEQ ID NO: 281 - protein, 12 aa, artificial sequence
  Synthetic construct
NLPRYQPPCKPL

SEQ ID NO: 282 - protein, 12 aa, artificial sequence
  Synthetic construct
RPPWKKPIPPSE

SEQ ID NO: 283 - protein, 12 aa, artificial sequence
  Synthetic construct
RQRPKDHFFSRP

SEQ ID NO: 284 - protein, 12 aa, artificial sequence
  Synthetic construct
SVPNKXVTVDGX

SEQ ID NO: 285 - protein, 12 aa, artificial sequence
  Synthetic construct
TTKWRHRAPVSP

SEQ ID NO: 286 - protein, 12 aa, artificial sequence
  Synthetic construct
WLGKNRIKPRAS

SEQ ID NO: 287 - protein, 12 aa, artificial sequence
  Synthetic construct
SNFKTPLPLTQS

SEQ ID NO: 288 - protein, 12 aa, artificial sequence
  Synthetic construct
SVSVGMKPSPRP

SEQ ID NO: 289 - protein, 7 aa, artificial sequence
  Synthetic construct
DLHTVYH

SEQ ID NO: 290 - protein, 7 aa, artificial sequence
  Synthetic construct
HIKPPTR

SEQ ID NO: 291 - protein, 7 aa, artificial sequence
  Synthetic construct
HPVWPAI

SEQ ID NO: 292 - protein, 7 aa, artificial sequence
  Synthetic construct
MPLYYLQ

SEQ ID NO: 293 - protein, 26 aa, artificial sequence
  Synthetic construct
HLTVPWRGGGSAVPFYSHSQITLPNH

SEQ ID NO: 294 - protein, 41 aa, artificial sequence
  Synthetic construct
GPHDTSSGGVRPNLHHTSKKEKRENRKVPFYSHSVTSRGNV

SEQ ID NO: 295 - protein, 7 aa, artificial sequence
  Synthetic construct
KHPTYRQ

SEQ ID NO: 296 - protein, 7 aa, artificial sequence
  Synthetic construct
HPMSAPR

SEQ ID NO: 297 - protein, 7 aa, artificial sequence
  Synthetic construct
MPKYYLQ

SEQ ID NO: 298 - protein, 7 aa, artificial sequence
  Synthetic construct
MHAHSIA

SEQ ID NO: 299 - protein, 12 aa, artificial sequence
  Synthetic construct
AKPISQHLQRGS

SEQ ID NO: 300 - protein, 12 aa, artificial sequence
  Synthetic construct
APPTPAAASATT

SEQ ID NO: 301 - protein, 12 aa, artificial sequence
  Synthetic construct
DPTEGARRTIMT

SEQ ID NO: 302 - protein, 12 aa, artificial sequence
  Synthetic construct
LDTSFPPVPFHA

SEQ ID NO: 303 - protein, 12 aa, artificial sequence
  Synthetic construct
LDTSFHQVPFHQ

SEQ ID NO: 304 - protein, 11 aa, artificial sequence
  Synthetic construct
LPRIANTWSPS

SEQ ID NO: 305 - protein, 12 aa, artificial sequence
  Synthetic construct
RTNAADHPAAVT

SEQ ID NO: 306 - protein, 12 aa, artificial sequence
  Synthetic construct
SLNWVTIPGPKI

SEQ ID NO: 307 - protein, 12 aa, artificial sequence
  Synthetic construct
TDMQAPTKSYSN

SEQ ID NO: 308 - protein, 12 aa, artificial sequence
  Synthetic construct
TIMTKSPSLSCG

SEQ ID NO: 309 - protein, 12 aa, artificial sequence
  Synthetic construct
TPALDGLRQPLR

SEQ ID NO: 310 - protein, 12 aa, artificial sequence
  Synthetic construct
TYPASRLPLLAP

SEQ ID NO: 311 - protein, 12 aa, artificial sequence
  Synthetic construct
AKTHKHPAPSYS

SEQ ID NO: 312 - protein, 12 aa, artificial sequence
  Synthetic construct
TDPTPFSISPER

SEQ ID NO: 313 - protein, 12 aa, artificial sequence
  Synthetic construct
SQNWQDSTSYSN

SEQ ID NO: 314 - protein, 12 aa, artificial sequence
  Synthetic construct
WHDKPQNSSKST

SEQ ID NO: 315 - protein, 12 aa, artificial sequence
  Synthetic construct
LDVESYKGTSMP

SEQ ID NO: 316 - protein, 7 aa, artificial sequence
  Synthetic construct
NTPKENW

SEQ ID NO: 317 - protein, 7 aa, artificial sequence
  Synthetic construct
NTPASNR

SEQ ID NO: 318 - protein, 7 aa, artificial sequence
  Synthetic construct
PRGMLST

SEQ ID NO: 319 - protein, 7 aa, artificial sequence
  Synthetic construct
PPTYLST

SEQ ID NO: 320 - protein, 12 aa, artificial sequence
  Synthetic construct
TIPTHRQHDYRS

SEQ ID NO: 321 - protein, 7 aa, artificial sequence
  Synthetic construct
TPPTHRL

SEQ ID NO: 322 - protein, 7 aa, artificial sequence
  Synthetic construct
LPTMSTP

SEQ ID NO: 323 - protein, 7 aa, artificial sequence
  Synthetic construct
LGTNSTP

SEQ ID NO: 324 - protein, 12 aa, artificial sequence
  Synthetic construct
TPLTGSTNLLSS

SEQ ID NO: 325 - protein, 7 aa, artificial sequence
  Synthetic construct
TPLTKET

SEQ ID NO: 326 - protein, 7 aa, artificial sequence
  Synthetic construct
KQSHNPP

SEQ ID NO: 327 - protein, 7 aa, artificial sequence
  Synthetic construct
QQSHNPP

SEQ ID NO: 328 - protein, 7 aa, artificial sequence
  Synthetic construct
TQPHNPP

SEQ ID NO: 329 - protein, 12 aa, artificial sequence
  Synthetic construct
STNLLRTSTVHP

SEQ ID NO: 330 - protein, 12 aa, artificial sequence
  Synthetic construct
HTQPSYSSTNLF

SEQ ID NO: 331 - protein, 7 aa, artificial sequence
  Synthetic construct
SLLSSHA

SEQ ID NO: 332 - protein, 12 aa, artificial sequence
  Synthetic construct
QQSSISLSSHAV

SEQ ID NO: 333 - protein, 7 aa, artificial sequence
  Synthetic construct
NASPSSL

SEQ ID NO: 334 - protein, 7 aa, artificial sequence
  Synthetic construct
HSPSSLR

SEQ ID NO: 335 - protein, 7 aa, artificial sequence
  Synthetic construct
KXSHHTH

SEQ ID NO: 336 - protein, 7 aa, artificial sequence
  Synthetic construct
EXSHHTH

SEQ ID NO: 337 - protein, 12 aa, artificial sequence
  Synthetic construct
SHHTHYGQPGPV

SEQ ID NO: 338 - protein, 7 aa, artificial sequence
  Synthetic construct
LESTSLL

SEQ ID NO: 339 - protein, 7 aa, artificial sequence
  Synthetic construct
DLTLPFH

SEQ ID NO: 340 - protein, 8 aa, artificial sequence
  Synthetic construct
RTNAADHP

SEQ ID NO: 341 - protein, 12 aa, artificial sequence
  Synthetic construct
IPWWNIRAPLNA

SEQ ID NO: 342 - protein, 18 aa, artificial sequence
  Synthetic construct
EQISGSLVAAPWEGEGER

SEQ ID NO: 343 - protein, 12 aa, artificial sequence
  synthetic hair-binding peptide
TPPELLHGAPRS

SEQ ID NO: 344 - protein, 18 aa, artificial sequence
  Synthetic construct
LDTSFHQVPFHQKRKRKD

SEQ ID NO: 345 - protein, 18 aa, artificial sequence
  Synthetic construct
EQISGSLVAAPWKRKRKD

SEQ ID NO: 346 - protein, 18 aa, artificial sequence
  Synthetic construct
TPPELLHGDPRSKRKRKD

SEQ ID NO: 347 - protein, 13 aa, artificial sequence
  Synthetic construct
NTSQLSTEGEGED

SEQ ID NO: 348 - protein, 13 aa, artificial sequence
  Synthetic construct
TPPELLHGDPRSC

SEQ ID NO: 349 - protein, 20 aa, artificial sequence
  synthetic hair-binding peptide
HINKTNPHQGNHHSEKTQRQ

SEQ ID NO: 350 - protein, 15 aa, artificial sequence
  Synthetic construct
HAHKNQKETHQRHAA

SEQ ID NO: 351 - protein, 15 aa, artificial sequence
  Synthetic construct
HEHKNQKETHQRHAA

SEQ ID NO: 352 - protein, 20 aa, artificial sequence
  Synthetic construct
HNHMQERYTEPQHSPSVNGL

SEQ ID NO: 353 - protein, 17 aa, artificial sequence
  Synthetic construct
THSTHNHGSPRHTNADA

SEQ ID NO: 354 - protein, 20 aa, artificial sequence
  synthetic hair-binding peptide
GSCVDTHKADSCVANNGPAT

SEQ ID NO: 355 - protein, 20 aa, artificial sequence
  synthetic hair-binding peptide
AQSQLPDKHSGLHERAPQRY

SEQ ID NO: 356 - protein, 20 aa, artificial sequence
  Synthetic construct
AQSQLPAKHSGLHERAPQRY

SEQ ID NO: 357 - protein, 20 aa, artificial sequence
  Synthetic construct
AQSQLPEKHSGLHERAPQRY

SEQ ID NO: 358 - protein, 20 aa, artificial sequence
  synthetic hair-binding peptide
TDMMHNHSDNSPPHRRSPRN

SEQ ID NO: 359 - protein, 20 aa, artificial sequence
  synthetic hair-binding peptide
TPPELAHTPHHLAQTRLTDR

SEQ ID NO: 360 - protein, 12 aa, artificial sequence
  Synthetic construct
RLLRLLRLLRLL

SEQ ID NO: 361 - protein, 12 aa, artificial sequence
  Synthetic construct
TPPELLHGEPRS

SEQ ID NO: 362 - protein, 12 aa, artificial sequence
  Synthetic construct
TPPELLHGAPRS

SEQ ID NO: 363 - protein, 12 aa, artificial sequence
  Synthetic construct
EQISGSLVAAPW

SEQ ID NO: 364 - protein, 12 aa, artificial sequence
  Synthetic construct
NEVPARNAPWLV

SEQ ID NO: 365 - protein, 13 aa, artificial sequence
  Synthetic construct
NSPGYQADSVAIG

SEQ ID NO: 366 - protein, 12 aa, artificial sequence
  Synthetic construct
AKPISQHLQRGS

SEQ ID NO: 367 - protein, 12 aa, artificial sequence
  Synthetic construct
LDTSFPPVPFHA

SEQ ID NO: 368 - protein, 12 aa, artificial sequence
  Synthetic construct
SLNWVTIPGPKI

SEQ ID NO: 369 - protein, 12 aa, artificial sequence
  Synthetic construct
TQDSAQKSPSPL

SEQ ID NO: 370 - protein, 12 aa, artificial sequence
  Synthetic construct
KELQTRNVVQRE

SEQ ID NO: 371 - protein, 12 aa, artificial sequence
  Synthetic construct
QRNSPPAMSRRD

SEQ ID NO: 372 - protein, 12 aa, artificial sequence
  Synthetic construct
TPTANQFTQSVP

SEQ ID NO: 373 - protein, 12 aa, artificial sequence
  Synthetic construct
AAGLSQKHERNR

SEQ ID NO: 374 - protein, 12 aa, artificial sequence
  Synthetic construct
ETVHQTPLSDRP

SEQ ID NO: 375 - protein, 12 aa, artificial sequence
  Synthetic construct
KNFPQQKEFPLS

SEQ ID NO: 376 - protein, 12 aa, artificial sequence
  Synthetic construct
LPALHIQRHPRM

SEQ ID NO: 377 - protein, 12 aa, artificial sequence
  Synthetic construct
QPSHSQSHNLRS

SEQ ID NO: 378 - protein, 12 aa, artificial sequence
  Synthetic construct
RGSQKSKPPRPP

SEQ ID NO: 379 - protein, 12 aa, artificial sequence
  Synthetic construct
THTQKTPLLYYH

SEQ ID NO: 380 - protein, 12 aa, artificial sequence
  Synthetic construct
TKGSSQAILKST

SEQ ID NO: 381 - protein, 7 aa, artificial sequence
  Synthetic construct
TAATTSP

SEQ ID NO: 382 - protein, 7 aa, artificial sequence
  Synthetic construct
LGIPQNL

SEQ ID NO: 383 - protein, 20 aa, artificial sequence
  Synthetic construct
THSTHNHGSPRHTNADAGNP

SEQ ID NO: 384 - protein, 20 aa, artificial sequence
  Synthetic construct
QQHKVHHQNPDRSTQDAHHS

SEQ ID NO: 385 - protein, 15 aa, artificial sequence
  Synthetic construct
HHGTHHNATKQKNHV

SEQ ID NO: 386 - protein, 15 aa, artificial sequence
  Synthetic construct
STLHKYKSQDPTPHH

SEQ ID NO: 387 - protein, 12 aa, artificial sequence
  Synthetic construct
SVSVGMKPSPRP

SEQ ID NO: 388 - protein, 12 aa, artificial sequence
  synthetic hair-binding peptide
TPPTNVLMLATK

SEQ ID NO: 389 - protein, 12 aa, artificial sequence
  Synthetic construct
TPPELLHGDPRS

SEQ ID NO: 390 - protein, 7 aa, artificial sequence
  synthetic hair-binding peptide
NTSQLST

SEQ ID NO: 391 - protein, 15 aa, artificial sequence
  Synthetic construct
STLHKYKSQDPTPHH

SEQ ID NO: 392 - protein, 12 aa, artificial sequence
  synthetic hair-binding peptide
GMPAMHWIHPFA

SEQ ID NO: 393 - protein, 15 aa, artificial sequence
  synthetic hair-binding peptide
HDHKNQKETHQRHAA

SEQ ID NO: 394 - protein, 20 aa, artificial sequence
  Synthetic construct
HNHMQERYTDPQHSPSVNGL

SEQ ID NO: 395 - protein, 20 aa, artificial sequence
  synthetic hair-binding peptide
TAEIQSSKNPNPHPQRSWTN

SEQ ID NO: 396 - protein, 12 aa, artificial sequence
  Synthetic construct
KRGRHKRPKRHK

SEQ ID NO: 397 - protein, 7 aa, artificial sequence
  Synthetic construct
RLLRLLR

SEQ ID NO: 398 - protein, 12 aa, artificial sequence
  Synthetic construct
HKPRGGRKKALH

SEQ ID NO: 399 - protein, 18 aa, artificial sequence
  Synthetic construct
KPRPPHGKKHRPKHRPKK

SEQ ID NO: 400 - protein, 18 aa, artificial sequence
  Synthetic construct
RGRPKKGHGKRPGHRARK

SEQ ID NO: 401 - protein, 12 aa, artificial sequence
  Synthetic construct
TPFHSPENAPGS

SEQ ID NO: 402 - protein, 13 aa, artificial sequence
  Synthetic construct
TPFHSPENAPGSK

SEQ ID NO: 403 - protein, 16 aa, artificial sequence
  Synthetic construct
TPFHSPENAPGSGGGS

SEQ ID NO: 404 - protein, 17 aa, artificial sequence
  Synthetic construct
TPFHSPENAPGSGGGSS

SEQ ID NO: 405 - protein, 15 aa, artificial sequence
  Synthetic construct
TPFHSPENAPGSGGG

SEQ ID NO: 406 - protein, 7 aa, artificial sequence
  Synthetic construct
FTQSLPR

SEQ ID NO: 407 - protein, 12 aa, artificial sequence
  Synthetic construct
KQATFPPNPTAY

SEQ ID NO: 408 - protein, 12 aa, artificial sequence
  Synthetic construct
HGHMVSTSQLSI

SEQ ID NO: 409 - protein, 7 aa, artificial sequence
  Synthetic construct
LSPSRMK

SEQ ID NO: 410 - protein, 7 aa, artificial sequence
  Synthetic construct
LPIPRMK

SEQ ID NO: 411 - protein, 7 aa, artificial sequence
  Synthetic construct
HQRPYLT

SEQ ID NO: 412 - protein, 7 aa, artificial sequence
  Synthetic construct
FPPLLRL

SEQ ID NO: 413 - protein, 7 aa, artificial sequence
  Synthetic construct
QATFMYN

SEQ ID NO: 414 - protein, 11 aa, artificial sequence
  Synthetic construct
VLTSQLPNHSM

SEQ ID NO: 415 - protein, 7 aa, artificial sequence
  Synthetic construct
HSTAYLT

SEQ ID NO: 416 - protein, 12 aa, artificial sequence
  Synthetic construct
APQQRPMKTFNT

SEQ ID NO: 417 - protein, 12 aa, artificial sequence
  Synthetic construct
APQQRPMKTVQY

SEQ ID NO: 418 - protein, 7 aa, artificial sequence
  Synthetic construct
PPWLDLL

SEQ ID NO: 419 - protein, 7 aa, artificial sequence
  Synthetic construct
PPWTFPL

SEQ ID NO: 420 - protein, 7 aa, artificial sequence
  Synthetic construct
SVTHLTS

SEQ ID NO: 421 - protein, 7 aa, artificial sequence
  Synthetic construct
VITRLTS

SEQ ID NO: 422 - protein, 12 aa, artificial sequence
  Synthetic construct
DLKPPLLALSKV

SEQ ID NO: 423 - protein, 12 aa, artificial sequence
  Synthetic construct
SHPSGALQEGTF

SEQ ID NO: 424 - protein, 12 aa, artificial sequence
  Synthetic construct
FPLTSKPSGACT

SEQ ID NO: 425 - protein, 12 aa, artificial sequence
  Synthetic construct
DLKPPLLALSKV

SEQ ID NO: 426 - protein, 7 aa, artificial sequence
  Synthetic construct
PLLALHS

SEQ ID NO: 427 - protein, 7 aa, artificial sequence
  Synthetic construct
VPISTQI

SEQ ID NO: 428 - protein, 12 aa, artificial sequence
  Synthetic construct
YAKQHYPISTFK

SEQ ID NO: 429 - protein, 7 aa, artificial sequence
  Synthetic construct
HSTAYLT

SEQ ID NO: 430 - protein, 12 aa, artificial sequence
  Synthetic construct
STAYLVAMSAAP

SEQ ID NO: 431 - protein, 12 aa, artificial sequence
  Synthetic construct
SVSVGMKPSPRP

SEQ ID NO: 432 - protein, 12 aa, artificial sequence
  Synthetic construct
TMGFTAPRFPHY

SEQ ID NO: 433 - protein, 12 aa, artificial sequence
  Synthetic construct
NLQHSVGTSPVW

SEQ ID NO: 434 - protein, 15 aa, artificial sequence
  Synthetic construct
QLSYHAYPQANHHAP

SEQ ID NO: 435 - protein, 12 aa, artificial sequence
  Synthetic construct
NQAASITKRVPY

SEQ ID NO: 436 - protein, 14 aa, artificial sequence
  Synthetic construct
SGCHLVYDNGFCDH

SEQ ID NO: 437 - protein, 14 aa, artificial sequence
  Synthetic construct
ASCPSASHADPCAH

SEQ ID NO: 438 - protein, 14 aa, artificial sequence
  Synthetic construct
NLCDSARDSPRCKV

SEQ ID NO: 439 - protein, 12 aa, artificial sequence
  Synthetic construct
NHSNWKTAADFL

SEQ ID NO: 440 - protein, 12 aa, artificial sequence
  Synthetic construct
GSSTVGRPLSYE

SEQ ID NO: 441 - protein, 12 aa, artificial sequence
  Synthetic construct
SDTISRLHVSMT

SEQ ID NO: 442 - protein, 12 aa, artificial sequence
  Synthetic construct
SPLTVPYERKLL

SEQ ID NO: 443 - protein, 12 aa, artificial sequence
  Synthetic construct
SPYPSWSTPAGR

SEQ ID NO: 444 - protein, 12 aa, artificial sequence
  Synthetic construct
VQPITNTRYEGG

SEQ ID NO: 445 - protein, 12 aa, artificial sequence
  Synthetic construct
WPMHPEKGSRWS

SEQ ID NO: 446 - protein, 14 aa, artificial sequence
  Synthetic construct
DACSGNGHPNNCDR

SEQ ID NO: 447 - protein, 14 aa, artificial sequence
  Synthetic construct
DHCLGRQLQPVCYP

SEQ ID NO: 448 - protein, 14 aa, artificial sequence
  Synthetic construct
DWCDTIIPGRTCHG

SEQ ID NO: 449 - protein, 12 aa, artificial sequence
  Synthetic construct
ALPRIANTWSPS

SEQ ID NO: 450 - protein, 12 aa, artificial sequence
  Synthetic construct
YPSFSPTYRPAF

SEQ ID NO: 451 - protein, 20 aa, artificial sequence
  Synthetic construct
AHPESLGIKYALDGNSDPHA

SEQ ID NO: 452 - protein, 20 aa, artificial sequence
  Synthetic construct
ASVSNYPPIHHLATSNTTVN

SEQ ID NO: 453 - protein, 14 aa, artificial sequence
  Synthetic construct
DECMEPLNAAHCWR

SEQ ID NO: 454 - protein, 14 aa, artificial sequence
  Synthetic construct
DECMHGSDVEFCTS

SEQ ID NO: 455 - protein, 14 aa, artificial sequence
  Synthetic construct
DLCSMQMMNTGCHY

SEQ ID NO: 456 - protein, 14 aa, artificial sequence
  Synthetic construct
DLCSSPSTWGSCIR

SEQ ID NO: 457 - protein, 20 aa, artificial sequence
  Synthetic construct
DPNESNYENATTVSQPTRHL

SEQ ID NO: 458 - protein, 20 aa, artificial sequence
  Synthetic construct
EPTHPTMRAQMHQSLRSSSP

SEQ ID NO: 459 - protein, 20 aa, artificial sequence
  Synthetic construct
GNTDTTPPNAVMEPTVQHKW

SEQ ID NO: 460 - protein, 15 aa, artificial sequence
  Synthetic construct
NGPDMVQSVGKHKNS

SEQ ID NO: 461 - protein, 15 aa, artificial sequence
  Synthetic construct
NGPEVRQIPANFEKL

SEQ ID NO: 462 - protein, 20 aa, artificial sequence
  Synthetic construct
NNTSADNPPETDSKHHLSMS

SEQ ID NO: 463 - protein, 20 aa, artificial sequence
  Synthetic construct
NNTWPEGAGHTMPSTNIRQA

SEQ ID NO: 464 - protein, 20 aa, artificial sequence
  Synthetic construct
NPTATPHMKDPMHSNAHSSA

SEQ ID NO: 465 - protein, 20 aa, artificial sequence
  Synthetic construct
NPTDHIPANSTNSRVSKGNT

SEQ ID NO: 466 - protein, 15 aa, artificial sequence
  Synthetic construct
NPTDSTHMMHARNHE

SEQ ID NO: 467 - protein, 14 aa, artificial sequence
  Synthetic construct
QHCITERLHPPCTK

SEQ ID NO: 468 - protein, 14 aa, artificial sequence
  Synthetic construct
TPCAPASFNPHCSR

SEQ ID NO: 469 - protein, 14 aa, artificial sequence
  Synthetic construct
TPCATYPHFSGCRA

SEQ ID NO: 470 - protein, 20 aa, artificial sequence
  Synthetic construct
WCTDFCTRSTPTSTSRSTTS

SEQ ID NO: 471 - protein, 20 aa, artificial sequence
  Synthetic construct
APPLKTYMQERELTMSQNKD

SEQ ID NO: 472 - protein, 20 aa, artificial sequence
  Synthetic construct
EPPTRTRVNNHTVTVQAQQH

SEQ ID NO: 473 - protein, 14 aa, artificial sequence
  Synthetic construct
GYCLRGDEPAVCSG

SEQ ID NO: 474 - protein, 20 aa, artificial sequence
  Synthetic construct
LSSKDFGVTNTDQRTYDYTT

SEQ ID NO: 475 - protein, 14 aa, artificial sequence
  Synthetic construct
NFCETQLDLSVCTV

SEQ ID NO: 476 - protein, 14 aa, artificial sequence
  Synthetic construct
NTCQPTKNATPCSA

SEQ ID NO: 477 - protein, 20 aa, artificial sequence
  Synthetic construct
PSEPERRDRNIAANAGRFNT

SEQ ID NO: 478 - protein, 18 aa, artificial sequence
  Synthetic construct
THNMSHFPPSGHPKRTAT

SEQ ID NO: 479 - protein, 14 aa, artificial sequence
  Synthetic construct
TTCPTMGTYHVCWL

SEQ ID NO: 480 - protein, 20 aa, artificial sequence
  Synthetic construct
YCADHTPDPANPNKICGYSH

SEQ ID NO: 481 - protein, 20 aa, artificial sequence
  Synthetic construct
AANPHTEWDRDAFQLAMPPK

SEQ ID NO: 482 - protein, 20 aa, artificial sequence
  Synthetic construct
DLHPMDPSNKRPDNPSDLHT

SEQ ID NO: 483 - protein, 14 aa, artificial sequence
  Synthetic construct
ESCVSNALMNQCIY

SEQ ID NO: 484 - protein, 20 aa, artificial sequence
  Synthetic construct
HNKADSWDPDLPPHAGMSLG

SEQ ID NO: 485 - protein, 20 aa, artificial sequence
  Synthetic construct
LNDQRKPGPPTMPTHSPAVG

SEQ ID NO: 486 - protein, 14 aa, artificial sequence
  Synthetic construct
NTCATSPNSYTCSN

SEQ ID NO: 487 - protein, 14 aa, artificial sequence
  Synthetic construct
SDCTAGLVPPLCAT

SEQ ID NO: 488 - protein, 20 aa, artificial sequence
  Synthetic construct
TIESSQHSRTHQQNYGSTKT

SEQ ID NO: 489 - protein, 20 aa, artificial sequence
  Synthetic construct
VGTMKQHPTTTQPPRVSATN

SEQ ID NO: 490 - protein, 20 aa, artificial sequence
  Synthetic construct
YSETPNDQKPNPHYKVSGTK